(19) United States
(12) Patent Application Publication
(10) Pub. No.: US 2009/0028842 A1
(43) Pub. Date: Jan. 29, 2009

(54) Dekkera/Brettanomyces Cytosine Deaminases And Their Use

(75) Inventors: Zoran Gojkovic, Holte (DK); Peter Kristoffersen, Horsholm (DK)

Correspondence Address: BROWDY AND NEIMARK, P.L.L.C.;624 NINTH STREET, NW, SUITE 300, WASHINGTON, DC, 20001-5303, US

(73) Assignee: ZGene A/S, Hørsholm (DK)

(21) Appl. No.: 12/088,031

(22) PCT Filed: Sep. 29, 2006
(86) PCT No.: PCT/DK2006/000539

§ 371 Date: Mar. 25, 2008

Related U.S. Application Data
(60) Provisional application No. 60/722,042, filed on Sep. 30, 2005.

(30) Foreign Application Priority Data
Sep. 30, 2005 (DK) PA 2005 01376

(51) Int. Cl. A61K 38/50; C12N 9/78; C12N 15/55; C12N 15/85; C07K 16/40; C12C 11/00; A61K 31/711; C12G 1/00; C12N 15/74; C12N 1/21; C12N 5/10

(52) U.S. Cl. 424/94.6; 435/227; 536/23.2; 435/252.3; 530/387.9; 426/15; 426/16; 426/11; 435/320.1; 435/375; 514/44

(57) ABSTRACT

The present invention relates to cytosine deaminase protein and cDNA from various species of the yeast genus Dekkera/Brettanomyces. Compared to yeast cytosine deaminase the novel cytosine deaminases are more efficient and have a higher stability. The invention also relates to the field of suicide gene therapy based on activation of a non-toxic prodrug, 5-fluorocytosine to a toxic drug 5-fluorouracil based on the enzymatic activity of novel cytosine deaminses. Finally the invention provides use of 5-fluorocytosine for controlling the growth of Dekkera/Brettanomyces yeast.

The present application claims the benefit of U.S. 60/722,042 filed 30 Sep. 2005, which is incorporated by reference in its entirety. It claims priority from Danish patent application no. PA 2005 01376, filed 30 Sep. 2005. All references cited in those applications and in the present application are hereby incorporated by reference in their entirety.

### TECHNICAL FIELD

The present invention relates to cytosine deaminase protein and cDNA from various species of the yeast genus Dekkera/Brettanomyces. The invention also relates to the field of suicide gene therapy based on activation of a non-toxic prodrug, 5-fluorocytosine to a toxic drug 5-fluorouracil based on the enzymatic activity of novel cytosine deaminses. Finally the invention relates to the use of 5-fluorocytosine for controlling the growth of Dekkera/Brettanomyces yeast.

### BACKGROUND

Cytosine deaminase (CD, cytosine aminohydrolase, EC 3.5.4.1) catalyzes the hydrolytic deamination of cytosine and 5-methylcytosine to uracil and thymine, respectively providing ammonia (Andersen, L., Kilstrup, M., and Neuhard, J. (1989) Arch. Microbiol. 152, 115-118

Kilstrup, M., Meng, L. M., Neuhard, J., and Nygaard, P. (1989) J. Bacteriol. 171, 2124-2127). The enzyme also deaminates the antifungal drug 5-fluorocytosine (5-FC) into highly toxic compound 5-fluorouracil (5-FU) which is further metabolized to several 5-fluoronucleotides that inhibit both RNA and DNA synthesis (Diasio, R. B. and Harris, B. E. (1989) Clin. Pharmacokinet. 16, 215-237). The CD gene is present in different prokaryotes and fungi, but a mammalian counterpart does not exist. Therefore 5-FC has relatively little toxicity for human cells and it seems that most of the toxicity observed with oral use of 5-FC in humans is due to deamination by intestinal bacteria (Diasio, R. B., Lakings, D. E., and Bennett, J. E. (1978) Antimicrob. Agents Chemother. 14, 903-908). This is utilized in gene directed enzyme prodrug activation therapy (GPAT) or so called suicide gene therapy where CD/5-FC is one of the most widely used enzyme/prodrug combinations for the treatment of cancers (Greco, O. and Dachs, G. U. (2001) J. Cell Physiol 187, 22-36). So far two different CD genes have been used, one is Escherichia coli CD, a hexamer of app. 300 kDa capable of deaminating a wide range of cytosine derivatives including 2-thiocytosine, 6-aza-cytosine, 4-azacytosine, and 5-FC (Porter, D. J. (2000) Biochim. Biophys. Acta 1476, 239-252). Another gene is Saccharomyces cerevisiae CD, which is a homodimer with a molecular mass of 35 kDa (Hayden, M. S., Linsley, P. S., Wallace, A. R., Marquardt, H., and Kerr, D. E. (1998) Protein Expr. Purif. 12, 173-184). The significant differences between the bacterial and yeast enzymes include not only size but also quaternary structure (Ireton, G. C., McDermott, G., Black, M. E., and Stoddard, B. L. (2002) J. Mol. Biol. 315, 687-697; Ireton, G. C., Black, M. E., and Stoddard, B. L. (2003) Structure. (Camb.) 11, 961-972) and relative substrate specificities and affinities. Yeast CD seems to be a superior candidate for suicide cancer therapy due to lower Kₘ for 5-FC (Kievit, E., Bershad, E., Ng, E., Sethna, P., Dev, I., Lawrence, T. S., and Rehemtulla, A. (1999) Cancer Res. 59, 1417-1421) and better thermal stability (Senter, P. D., Su, P. C., Katsuragi, T., Sakai, T., Cosand, W. L., Hellstrom, I., and Hellstrom, K. E. (1991) Bioconjug. Chem. 2, 447-451).

The majority of fungi express CD and are therefore able to grow on cytosine as the sole source of nitrogen. In contrast, very few yeasts can utilize uracil or its degradation products as nitrogen source (LaRue, T. A. and Spencer, J. F. (1968) Can. J. Microbiol. 14, 79-86). While S. cerevisiae cannot grow on uracil, relatively closely related S. kluyveri has a functional degradation pathway and can grow on both pyrimidines and purines (Gojkovic, Z., Paracchini, S., and Piskur, J. (1998) Adv. Exp. Med. Biol. 431, 475-479). Enzymes responsible for degradation of 5,6-dihydrouracil and N-carbamoyl-β-alanine have been characterized in this yeast (Gojkovic, Z., Jahnke, K., Schnackerz, K. D., and Piskur, J. (2000) J. Mol. Biol. 295, 1073-1087; Gojkovic, Z., Sandrini, M. P., and Piskur, J. (2001) Genetics 158, 999-1011), but catabolism of cytosine has not been studied in this or any other yeasts with exception of S. cerevisiae (Erbs, P., Exinger, F., and Jund, R. (1997) Curr. Genet. 31, 1-6) and some Candida species (Fasoli, M. O., Kerridge, D., Morris, P. G., and Torosantucci, A. (1990) Antimicrob. Agents Chemother. 34, 1996-2006; Hope, W. W., Tabernero, L., Denning, D. W., and Anderson, M. J. (2004) Antimicrob. Agents Chemother. 48, 4377-4386). The yeasts of the genus Brettanomyces, the anamorph form of the genus Dekkera, are well-known wine spoilage yeasts which produce undesirable off-flavours such as volatile phenols, acetic acid and tetrahydropyridines (van der Walt, J. P. and van Kerken, A. E., (1958) Antonie Van Leeuwenhoek 24, 241). Although these yeasts are not normally found on grapes and in fermenting must, they can develop at the end of the alcoholic fermentation and during wine ageing in wooden barrels. Brettanomyces includes five species: B. bruxellensis, B. anomalus, B. custersianus, B. naardenensis and B. nanus which was added following the renaming of Eeniella nana (Kurtzman, C. and Fell, J. W. The yeasts, a taxonomic study. 1998. Elsevier Science, 4th edition). Based on rDNA sequence homology it seems that this yeast group represents an evolutionary closely related and clearly separate clade with placement somewhere between Euascomycetes and Hemiascomycetes (Cai, J., Roberts, I. N., and Collins, M. D. (1996) Int. J. Syst. Bacteriol. 46, 542-549; Kurtzman, C. P. and Robnett, C. J. (1998) Antonie Van Leeuwenhoek 73, 331-371). Despite considerable industrial importance very limited molecular studies involving these yeasts exists. The main reason for this is lack of appropriate molecular tools as conventional yeast methods and vectors are not applicable for this genus. The majority of research on Brettanomyces/Dekkera focuses on early detection Cocolin, L., Rantsiou, K., lacumin, L., Zironi, R., and Comi, G. (2004) Appl. Environ. Microbiol. 70, 1347-1355; Phister, T. G. and Mills, D. A. (2003) Appl. Environ. Microbiol 69, 7430-7434; Stender, H., Kurtzman, C., Hyldig-Nielsen, J. J., Sorensen, D., Broomer, A., Oliveira, K., Perry-O'Keefe, H., Sage, A., Young, B., and Coull, J. (2001) Appl. Environ. Microbiol. 67, 938-941) or elimination of these yeasts from vine (Comitini, F., De Ingeniis, J., Pepe, L., Mannazzu, I., and Clani, M. (2004) FEMS Microbiol. Lett. 238, 235-240). Comitini and co-workers have suggested to eliminate Dekkera/Brettanomyces yeasts from wine by adding toxins from other yeasts (e.g. Pichia anomala and Kluyveromyces wickerhamii).

### SUMMARY OF THE INVENTION

In a first aspect the invention relates to an isolated cytosine deaminase (EC 3.5.4.1) selected from the group consisting of:

i. a cytosine deaminase derived from Dekkera/Brettanomyces,
ii. a cytosine deaminase comprising an amino acid sequence having at least 70% sequence identity to SEQ ID NO 2, 5 or 8, more preferably at least 75%, more preferably at least 80%, more preferably at least 85%, more preferably at least 90%, more preferably at least 95%, more preferably at least 97%; and
iii. a polypeptide fragment of any of i. through ii. possessing cytosine deaminase activity.

An isolated nucleic acid molecule selected from the group consisting of:

a. a nucleic acid comprising a cytosine deaminase open reading frame derived from a Dekkera/Brettanomyces species;
b. a nucleic acid comprising a nucleotide sequence being at least 70% identical to SEQ ID NO 1, 4, or 7;
c. a nucleic acid encoding a cytosine deaminase having at least 70% sequence identity to SEQ ID NO 2, 5, or 8;
d. a nucleic acid encoding a cytosine deaminase and being capable of hybridising to a nucleic acid molecule having the complementary sequence of SEQ ID NO 1, 4, or 7;
e. a fragment comprising at least 100 consecutive nucleotide bases of SEQ ID NO 1, 4 or 7; and
f. a subsequence of any of a through d encoding a cytosine deaminase.

In a further aspect the invention relates to a vector comprising a nucleic acid according to the invention, and to an isolated host cell transfected or transduced with the expression vector of the invention.

In a still further aspect the invention relates to a process for producing a Dekkera/Brettanomyces cytosine deaminase according to the invention comprising culturing a host cell according to the invention in vitro and recovering the expressed cytosine deaminase from the culture.

Furthermore, the invention relates to a packaging cell line capable of producing an infective vector particle, said vector particle comprising a virally derived genome comprising a 5′ viral LTR, a tRNA binding site, a packaging signal, a promoter operably linked to a polynucleotide sequence encoding a Dekkera/Brettanomyces cytosine deaminase according to the invention; an origin of second strand DNA synthesis, and a 3′ viral LTR. Preferably the vector particle is replication defective.

In a further aspect the invention relates to the use of the polypeptide of the invention, the nucleic acid of the invention, or the expression vector of the invention for the preparation of a medicament. Preferably the medicament is for the treatment of cancer.

In a further aspect, the invention relates to a pharmaceutical composition comprising the polypeptide of the invention, the nucleic acid of the invention, or the expression vector of the invention and a pharmaceutically acceptable diluent, carrier or excipient.

In a preferred embodiment the composition further comprises 5-fluorocytosine for simultaneous, separate or successive administration in cancer therapy

In a further aspect the invention relates to a method of treatment of cancer comprising administering to a patient inflicted with cancer a therapeutically effective amount of a Dekkera/Brettanomyces cytosine deaminase according to the invention and a therapeutically effective amount of 5-FC.

Furthermore the invention relates to a method of sensitising a mammalian cell to 5-fluorocytosine comprising transfecting or transducing said cell with an expression vector according to the invention, and delivering 5-fluorocytosine to said cell.

The polynucleotide sequence encoding a Dekkera/Brettanomyces CD according to the invention may also be used as a selection marker in molecular biology.

Furthermore, the invention relates to a method for deaminating a cytosine derivative comprising exposing said cytosine derivative to a cytosine deaminase according to the invention and recovering the deaminated cytosine derivative.

In a still further aspect the invention relates to the use of 5-fluorocytosine for controlling the growth of Dekkera/Brettanomyces.

### BRIEF DESCRIPTION OF THE DRAWINGS

FIG. 1. Growth of D. bruxellensis and S. cerevisiae after 5 days on plates containing cytosine and 5-FC. Growth inhibition of D. bruxellensis was observed already at 0.1 μM of 5-FC, while at 0.36 μM of 5-FC there was no visible growth. S. cerevisiae growth was first inhibited by addition of 1 μM of 5-FC.

FIG. 2. Growth of various Dekkera/Brettanomyces strains after 5 days on plates containing cytosine and 5-FC. Growth inhibition of all Dekkera and Brettanomyces species was observed at 0.1 or 0.36 μM 5-FC. Under these conditions growth of control strains S. cerevisiae and Metschnikowia reukauffii was not inhibited.

FIG. 3. Genetic structure of fungal CD genes. While S. cerevisiae CD is without introns, C. albicans CD contains one intron. D. bruxellensis CD has two introns located at the beginning and in the middle of the gene.

FIG. 4. Alignment of S. cerevisiae and D. bruxellensis CDs. The comparison was assembled with the ClustalX 1.81 program, Boxshade depicts all identical amino acids in white on black and similar amino acids are black on grey. The residues involved in the active center of S. cerevisiae CD are marked by ▴, while residues responsible for thermo stability of the enzyme are marked by ▪.

FIG. 5. Alignment of D. anomala, B. custersianus and D. bruxellensis CDs. The comparison was assembled with the ClustalX 1.81 program, Boxshade depicts all identical amino acids in white on black and similar amino acids are black on grey. D. anomala, B. custersianus CDs are partial sequences missing app. 16 amino acids at N terminus.

FIG. 6. Alignment of C. albicans (Acc. nr. AAC15782), D. anomala (partial sequence) and S. cerevisiae (Acc. nr. U55193) CDs. The comparison was assembled with the ClustalX 1.81 program. Boxshade depicts all identical amino acids in white on black and similar amino acids are black on grey. Residues which may be responsible for thermostablity and superior properties of Dekkera/Brettanomyces CDs showing no similarity to S. cerevisiae and C. albicans CDs are marked by ▪.

FIG. 7. Protein gel graph generated by Agilent Bioanalyzer of purified yeast CDs. The first lane shows the molecular weight marker. Lane 2 shows S. cerevisiae CD and Lane 3 shows D. bruxellensis CD.

FIG. 8. Temperature activity of S. cerevisiae (PZG738, diamonds) and D. bruxellensis (PZG893, squares) cytosine deaminase measured at time intervals (hours of storage) at 50° C. (FIG. 8a) and at 37° C. (FIG. 8b). The Y-axis shows the enzyme activity in percent of the initial activity of D. bruxellensis cytosine deaminase.

FIG. 9. Dekkera bruxellensis cytosine deaminase transduction of a breast cancer cell line enhances toxicity of 5-FC. The x-axis shows the concentration of 5-FC in mM. The y-axis shows absorbence in relative values. MCF7 cell line was transduced with a retrovirus vector encoding D. bruxellensis cytosine deaminase (FIG. 9b) and “empty” vector respectively (FIG. 9a). Cells were exposed to increasing concentrations of 5-FC and cell killing was measured. IC₅₀ for cells transduced with empty vector was 9.29 mM and for cells transduced with cytosine deaminase from D. bruxellensis was 2.435 mM.

### DEFINITIONS

Cytosine deaminase. A cytosine deaminase is an enzyme having cytosine deaminase activity (EC 3.5.4.1). A cytosine deaminase may be abbreviated as CD.

Sequence identity. The level of sequence identity between a query and a subject sequence is preferably determined using a sequence alignment program, such as the ClustalX 1.81 program (Jeanmougin, F., Thompson, J. D., Gouy, M., Higgins, D. G., and Gibson, T. J. (1998) Trends Biochem. Sci. 23, 403-405). The two sequences are aligned using the standard settings of the program. The number of fully conserved residues is calculated and divided by the length of the query sequence.

The terms “fragment,” “derivative” and “analog” when referring to the polypeptide of SEQ ID No. 2, 5 or 8, means a polypeptide which retains essentially the same biological function or activity as such polypeptide.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

### Hybridisation Conditions:

Suitable experimental conditions for determining hybridisation at low, medium, or high stringency conditions, respectively, between a nucleotide probe and a homologous DNA or RNA sequence, involves pre-soaking of the filter containing the DNA fragments or RNA to hybridise in 5×SSC [Sodium chloride/Sodium citrate; cf. Sambrook et al.; Molecular Cloning: A Laboratory Manual, 2^{nd} Ed., Cold Spring Harbor Lab., Cold Spring Harbor, N.Y. 1989] for 10 minutes, and prehybridization of the filter in a solution of 5×SSC, 5×Denhardt's solution [cf. Sambrook et al.; Op cit.], 0.5% SDS and 100 μg/ml of denatured sonicated salmon sperm DNA [cf. Sambrook et al.,; Op cit.], followed by hybridisation in the same solution containing a concentration of 10 ng/ml of a random-primed [Feinberg A P & Vogelstein B; Anal. Biochem. 1983 132 6-13], ³²P-dCTP-labeled (specific activity>1×10⁹ cpm/μg) probe for 12 hours at approximately 45° C.

The filter is then washed twice for 30 minutes in 2×SSC, 0.5% SDS at a temperature of at least 55° C. (low stringency conditions), more preferred of at least 60° C. (medium stringency conditions), still more preferred of at least 65° C. (medium/high stringency conditions), even more preferred of at least 70° C. (high stringency conditions), and yet more preferred of at least 75° C. (very high stringency conditions).

Molecules to which the oligonucleotide probe hybridises under these conditions may be labelled to detect hybridisation. The complementary nucleic acids or signal nucleic acids may be labelled by conventional methods known in the art to detect the presence of hybridised oligonucleotides. The most common method of detection is the use of autoradiography with e.g. ³H, ¹²⁵I, ³⁵S, ¹⁴C, or ³²P-labelled probes, which may then be detected using an x-ray film. Other labels include ligands, which bind to labelled antibodies, fluorophores, chemoluminescent agents, enzymes, or antibodies, which can then serve as specific binding pair members for a labelled ligand.

### DETAILED DESCRIPTION

### Dekkera Cytosine Deaminase

By the present invention there is provided isolated cytosine deaminases (EC 3.5.4.1) selected from the group consisting of:

i. a cytosine deaminase derived from Dekkera/Brettanomyces,
ii. a cytosine deaminase comprising an amino acid sequence having at least 70% sequence identity to SEQ ID NO 2, 5 or 8, more preferably at least 75%, more preferably at least 80%, more preferably at least 85%, more preferably at least 90%, more preferably at least 95%, more preferably at least 97%; and
iii. a polypeptide fragment of any of i. through ii. possessing cytosine deaminase activity.

The appended examples indicate that cytosine deaminases from Dekkera/Brettanomyces yeasts in general are superior to known yeast and bacterial cytosine deaminases in terms of converting 5-fluorocytosine to 5-fluoruracil. Due to the superior enzymatic properties, the enzymes are particularly efficient at converting the non-toxic prodrug 5-fluorocytosine into the highly toxic compound 5-fluorouracil. Downstream products of 5-fluorouracil inhibit RNA and DNA synthesis and therefore the compound is capable of killing in particular dividing cells, including cancer cells. Furthermore a higher stability of the cytosine deaminases of the present invention make these enzymes superior to known cytosine deaminases in particular for therapeutic use.

Specific methods for cloning of Dekkera/Brettanomyces cytosine deaminases had to be developed since no molecular and genetic systems existed for these species prior to the preset invention, and since the gene structure of Dekkera/Brettanomyces cytosine deaminases turned out to be different from the gene structure of yeast cytosine deaminases.

The examples provide the (partial or full length) cDNA and protein sequences of cytosine deaminases from D. bruxellensis, D. anomala and B. custersianus. The other species of the Dekkera/Brettanomyces genus also contain at least one gene coding for cytosine deaminase. As demonstrated in the examples, strains from Dekkera/Brettanomyces are particularly susceptible to 5-fluorocytosine as opposed to yeast strains from which cytosine deaminases have previously been isolated or sequenced. The present inventors therefore believe that cytosine deaminases from other Dekkera/Brettanomyces are superior to known yeast and/or bacterial cytosine deaminases in terms of activating 5-FC.

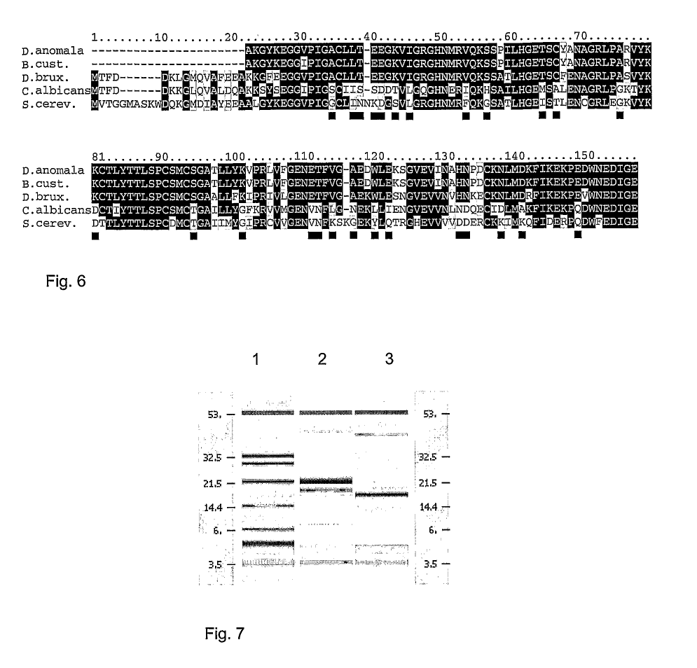

The high degree of conservation of cytosine deaminase within the Dekkera/Brettanomyces genus can be seen in the ClustalX 1.81 alignment in FIG. 5. It is visualised in FIG. 6 that the cytosine deaminses from this genus are distinct from the cytosine deaminases from other yeast is visualised. FIG. 6 shows an alignment of the full length protein sequence of D. bruxellensis cytosine deaminase and partial sequences from D. anomala and B. custersianus agains the cytosine deaminase from S. cerevisiae. Residues that are distinct for Dekkera/Brettanomyces cytosine deaminases and may be important for the enhanced properties of these enzymes compared to other yeast cytosine deaminases are marked with a black square.

Therefore, in one embodiment, the invention relates an isolated cytosine deaminase is derived from a Dekkera/Brettanomyces species. In a preferred embodiment the isolated cytosine deaminase has at least 70% sequence identity to SEQ ID No 2, which is the amino acid sequence of cytosine deaminase from Dekkera bruxellensis. More preferably the isolated cytosine deaminase has at least 75% sequence identity to SEQ ID NO 2, more preferably at least 80%, more preferably at least 85%, more preferably at least 90%, more preferably at least 95%, more preferably at least 97%.

In a preferred embodiment the isolated cytosine deaminase has at least 70% sequence identity to SEQ ID No 5, which is a partial amino acid sequence of cytosine deaminase from Dekkera anomala. More preferably the isolated cytosine deaminase has at least 75% sequence identity to SEQ ID NO 5, more preferably at least 80%, more preferably at least 85%, more preferably at least 90%, more preferably at least 95%, more preferably at least 97%.

In a preferred embodiment the isolated cytosine deaminase has at least 70% sequence identity to SEQ ID No 8, which is a partial amino acid sequence of cytosine deaminase from B. custersianus. More preferably the isolated cytosine deaminase has at least 75% sequence identity to SEQ ID NO 8, more preferably at least 80%, more preferably at least 85%, more preferably at least 90%, more preferably at least 95%, more preferably at least 97%.

The present invention further relates to isolated polypeptides which have the deduced amino acid sequence of SEQ ID No. 2, 5 or 8, as well as fragments, analogs and derivatives of such polypeptides.

The polypeptide of the present invention may be a recombinant polypeptide, a natural polypeptide or a synthetic polypeptide, preferably a recombinant polypeptide.

In a preferred embodiment the sequence variant of SEQ ID NO 2, 5, or 8 comprises those residues that have been marked with a black square in the alignment in FIG. 6. These residues make Dekkera/Brettanomyces cytosine deaminases distinct from other yeast deaminases.

In one embodiment, any amino acid in the CD polypeptide is changed to a different amino acid (compared to SEQ ID NO 2, 5 or 8), provided that no more than 15% of the amino acid residues in the sequence are so changed. More preferably no more than 10% of the amino acid residues are so changed, more preferably no more than 5% or the amino acid residues are so changed, more preferably no more than 5 amino acid residues are so changed.

Preferably the sequence variants are capable of converting 5-FC to 5-FU.

In a preferred embodiment of the invention the cytosine deaminase of the invention is capable of reducing the LD₁₀₀ of 5-FC by at least a factor of 2 compared to the LD₁₀₀ for S. cerevisiae when expressed in a cell. Preferably the cell is a bacterial or mammalian cell.

More preferably, the cell is a mammalian cell, such as a human cancer cell. More preferably the LD₁₀₀ is reduced by a factor of at least 4, even more preferably by a factor of at least 10.

The fragment, derivative or analog of the polypeptide of SEQ ID No. 2, 5 or 8 may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide, such as a leader or secretory sequence or a sequence which is employed for purification of the polypeptide. Such fragments, derivatives and analogs are deemed to be within the scope of those skilled in the art from the teachings herein.

Certain human tumour cells have a natural resistance to 5-FU. For use in such cells it is advantageous that the expressed protein also has uracil phosphoribosyltransferase activity. It has been shown that the sensitivity to 5-FC may be increased greatly (100-1000 times) by co-expressing a cytosine deaminase and a uracil phosphoribosyltransferase (WO 2004/061079; WO 96/16183; Erbs et al 2000, Cancer Res. 15; 60(14):3813-22). Therefore in one embodiment, the cytosine deaminase of the present invention is part of a fusion protein, wherein the other part comprises a uracil phosphoribosyltransferase. The UPRTase may be truncated in its N-terminal part (WO 99/54481). The UPRTase is preferably derived from yeast, such as S. cerevisiae (Kern et al, 1990, Gene 88:149-157). The UPRTase may also be derived from Candida kefyr (WO 2004/061079). Activity of a yeast CD may also be increased by adding the N-terminal of a UPRTase polypeptide (WO 2005/007957). Other references describing the simultaneous use of cytosine deaminase and UPRTase include: Seo E, Abei M, Wakayama M, Fukuda K, Ugai H, Murata T, Todoroki T, Matsuzaki Y, Tanaka N, Hamada H, Yokoyama K K. Cancer Res. 2005 Jan. 15; 65(2):546-52. “Effective gene therapy of biliary tract cancers by a conditionally replicative adenovirus expressing uracil phosphoribosyltransferase: significance of timing of 5-fluorouracil administration”; Porosnicu M, Mian A, Barber G N. Cancer Res. 2003 Dec. 1; 63(23):8366-76. “The oncolytic effect of recombinant vesicular stomatitis virus is enhanced by expression of the fusion cytosine deaminase/uracil phosphoribosyltransferase suicide gene”; Chung-Faye G A, Chen M J, Green N K, Burton A, Anderson D, Mautner V, Searle P F, Kerr D J. Gene Ther. 2001 October; 8(20):1547-54. “In vivo gene therapy for colon cancer using adenovirus-mediated, transfer of the fusion gene cytosine deaminase and uracil phosphoribosyltransferase”.

The polypeptides and polynucleotides of the present invention are preferably provided in an isolated form, and preferably are purified to homogeneity.

### Cytosine Deaminase Nucleic Acids

In another aspect the invention relates to an isolated nucleic acid molecule selected from the group consisting of:

a. a nucleic acid comprising a cytosine deaminase open reading frame derived from a Dekkera/Brettanomyces species;
b. a nucleic acid comprising a nucleotide sequence being at least 70% identical to SEQ ID NO 1, 4, or 7;
c. a nucleic acid encoding a cytosine deaminase having at least 70% sequence identity to SEQ ID NO 2, 5, or 8;
d. a nucleic acid encoding a cytosine deaminase and being capable of hybridising to a nucleic acid molecule having the complementary sequence of SEQ ID NO 1, 4, or 7;
e. a fragment comprising at least 100 consecutive nucleotide bases of SEQ ID NO 1, 4 or 7; and
f. a subsequence of any of a through d encoding a cytosine deaminase.

In one embodiment the nucleic acid of the invention comprises a cytosine deaminase open reading frame derived from a Dekkera/Brettanomyces species. The present invention provides the nucleic acid sequence of D. bruxellensis cytosine deaminase cDNA (SEQ ID NO 1) and genomic sequence (SEQ ID NO 3); D. anomala partial cytosine deaminase cDNA (SEQ ID No 4) and genomic sequence (SEQ ID NO 6); B. custersianus cytosine deaminase partial cDNA (SEQ ID NO 7) and genomic sequence (SEQ ID NO 9). Using these sequence information, it is possible to identify and clone the orthologous sequences from other species of the Dekkera/Brettanomyces genus.

Sequences from other Dekkera/Brettanomyces species may be identified and cloned in various ways. Partial sequences from D. anomala and B. custrianus have been identified the same way as D. bruxellensis (example 4). One method based on the identification of the CD promoter in Dekkera bruxellensis is described in Example 3 of the present application. Another method comprises the use of degenerate primers with optimum Dekkera codon usage. As the present invention represents the first cloning of an ORF from any species of Dekkera/Brettanomyces, there has been no prior knowledge of the codon usage within the genus. Now, primers with optimum codon usage can be designed and cytosine deaminase genes can be PCR cloned from other species of the genus. A further method includes Southern hybridisation using a fragment of the D. bruxellensis cytosine deaminase coding sequence. On the DNA level D. bruxellensis cytosine deaminase open reading frame has no significant sequence homology to known sequences. On the other hand, the percent sequence identity to a partial D. anomala cytosine deaminase is approximately 75%. Therefore, it is possible to identify and sequence cytosine deaminase genes from other species of the genus using the sequence information provided for the first time in the present application.

In another embodiment the nucleic acid of the invention comprises a nucleotide sequence being at least 70% identical to SEQ ID NO 1, 4 or 7, more preferably at least 75%, more preferably at least 80%, more preferably at least 85%, more preferably at least 90%, more preferably at least 95%, more preferably at least 97%. The coding sequence of D. bruxellensis CD (SEQ ID NO 1), D. anomala CD (SEQ ID NO 4) and B. custersianus (SEQ ID NO 7) may be changed due to the degeneracy of the genetic code and may also be changed without affecting the activity of the encoded polypeptide as it is known in the art that amino acid sequences may be mutated without affecting activity.

In a further embodiment the nucleic acid of the invention encodes a cytosine deaminase having at least 70% sequence identity to SEQ ID NO 2, 5 or 8, more preferably at least 75%, more preferably at least 80%, more preferably at least 85%, more preferably at least 90%, more preferably at least 95%, more preferably at least 97%.

In a still further embodiment of the invention, the nucleic acid of the invention encodes a cytosine deaminase and is capable of hybridising to a nucleic acid molecule having the complementary sequence of SEQ ID NO 1, 4, or 7 or a sub-sequence thereof. The hybridisation conditions preferably are as described in the definitions section of the present application. Sequences capable of hybridising to a sub-sequence of SEQ ID NO 1, 4, or 7 include cytosine deaminase nucleic acids from other species of the Dekkera/Brettanomyces genus. Preferably the hybridisation conditions are adjusted such that cytosine deaminase mRNAs or cDNAs from S. cerevisiae, C. albicans or C. kefyr do not hybridise. Preferably the hybridisation is under conditions of medium stringency, more preferably under conditions of high stringency.

Fragments of the full length of the Dekkera/Brettanomyces CD genes may be used as a hybridization probe for a cDNA library to isolate the full length CD genes and to isolate other genes which have a high sequence similarity to the Dekkera/Brettanomyces CD genes or similar biological activity. Probes of this type generally have at least 20 bases. Preferably, however, the probes have at least 30 bases and generally do not exceed 50 bases, although they may have a greater number of bases. The probe may also be used to identify a cDNA clone corresponding to a full length transcript and a genomic clone or clones that contain the complete Dekkera/Brettanomyces CD genes including regulatory and promoter regions, exons, and introns. An example of a screen comprises isolating the coding region of the Dekkera/Brettanomyces CD genes by using the known DNA sequence to synthesize an oligonucleotide probe. Labelled oligonucleotides having a sequence complementary to that of the gene of the present invention are used to screen a cDNA library, genomic DNA or mRNA to determine which members of the library the probe hybridizes to.

In a preferred embodiment, the nucleic acid is codon optimised for expression in human beings. This may lead to enhanced expression compared to the use of Dekkera codons. In another preferred embodiment the nucleic acid has a reduced CpG codon usage. The CpG codon usage may be reduced or completely eliminated.

In one embodiment the nucleic acid is operably fused to a nucleic acid encoding uracil phosphoribosyltransferase. As described above this may lead to enhanced cytotoxicity of 5-FC in certain human tumour cells. The uracil phosphoribosyltransferase may be derived from a yeast, preferably Saccharomyces cerevisiae or C. kefyr.

The polynucleotide of the present invention may be in the form of RNA or in the form of DNA, which DNA includes cDNA, genomic DNA, and synthetic DNA, or PNA or LNA. The DNA may be double-stranded or single-stranded, and if single stranded may be the coding strand or non-coding (anti-sense) strand. The coding sequence which encodes the polypeptide may be identical to the coding sequences shown in SEQ ID No. 1 and 3, 4 and 6, 7 and 9 or may be a different coding sequence which coding sequence, as a result of the redundancy or degeneracy of the genetic code, encodes the same polypeptide as the nucleic acid of SEQ ID No. 1, 4 or 7.

The polynucleotides which encode the polypeptide of SEQ ID No. 2, 5 or 8 may include: only the coding sequence for the polypeptide; the coding sequence for the polypeptide and additional coding sequence; the coding sequence for the polypeptide (and optionally additional coding sequence) and non-coding sequence, such as introns or non-coding sequence 5′ and/or 3′ of the coding sequence for the polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode fragments, analogues, mutants, and derivatives of the polypeptides having the deduced amino acid sequence of SEQ ID No. 2, 5 or 8.

Thus, the present invention includes polynucleotides encoding the same polypeptides as shown in SEQ ID No. 2, 5 or 8 as well as variants of such polynucleotides which variants encode a fragment, derivative, mutant, or analogue of the polypeptides of SEQ ID NO. 2, 5 or 8. Such nucleotide variants include deletion variants, substitution variants and addition or insertion variants.

The polynucleotide may have a coding sequence, which is a naturally occurring allelic variant of the coding sequence shown in SEQ ID No. 1, 4, or 7. As known in the art, an allelic variant is an alternate form of a polynucleotide sequence, which may have a substitution, deletion or addition of one or more nucleotides, which does not substantially alter the function of the encoded polypeptide.

The encoded CD when compared to cytosine deaminase from S. cerevisiae in a eukaryotic cell preferably decreases at least two fold the LD₁₀₀ of 5-FC. More preferably the LD₁₀₀ is decreased at least 4 fold, more preferably at least 10 fold.

The polynucleotides of the present invention may also have the coding sequence fused in frame to a tag sequence which allows for purification of the polypeptide of the present invention. The marker sequence may be a hexahistidine tag supplied by a pQE-9 vector to provide for purification of the polypeptide fused to the marker in the case of a bacterial host, or, for example the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (184)). In addition, a GST tag such as supplied by the pGEX-2T vector from Pharmacia can be used. Other tags include a FLAG tag.

### Vectors

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the CD genes. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies.

Suitable expression vectors may be a viral vector derived from Herpes simplex, adenovira, adenoassociated vira, lentivira, retrovira, or vaccinia vira, or from various bacterially produced plasmids, and may be used for in vivo delivery of nucleotide sequences to a whole organism or a target organ, tissue or cell population. Other delivery methods include, but are not limited to, liposome transfection, electroporation, transfection with carrier peptides containing nuclear or other localising signals, and gene delivery via slow-release systems.

Other suitable expression vectors include general purpose mammalian vectors which are also obtained from commercial sources (Invitrogen Inc., Clonetech, Promega, BD Biosecences, etc) and contain selection for Geneticin/neomycin (G418), hygromycin B, puromycin, Zeocin/bleomycin, blasticidin SI, mycophenolic acid or histidinol.

The expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vectors include the following classes of vectors: general eukaryotic expression vectors, vectors for stable and transient expression and epitag vectors as well as their TOPO derivatives for fast cloning of desired inserts (see list below for available vectors).

Ecdysone-Inducible Expression: pIND(SP1) Vector; pIND/V5-His Tag Vector Set;

pIND(SP1)/V5-His Tag Vector Set; EcR Cell Lines; Muristerone A. Stable Expression: pcDNA3.1/Hygro; pSecTag A, B & C; pcDNA3.1 (−)/MycHis A, B & C pcDNA3.1+/−; pcDNA3.1/Zeo (+) and pcDNA3.1/Zeo (−); pcDNA3.1/H is A, B, & C; pRc/CMV2; pZeoSV2 (+) and pZeoSV2 (−); pRc/RSV; pTracer™-CMV; pTracer™-SV40.

Transient Expression: pCDM8; pcDNA1.1; pcDNA1.1/Amp.

Epitag Vectors: pcDNA3.1/MycHis A, B & C; pcDNA3.1/V5-His A, B, & C.

Large numbers of suitable vectors and promoters are known to those of skill in the art, and are commercially available. The following vectors are provided by way of example. Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK2233, pKK233-3, pDR540, pRITS (Pharmacia). Eukaryotic: pWLNEO, pSV2CAT, pOG44, pXTI, pSG (Stratagene) pSVK3, pBPV, pMSG, pSVL (Pharmacia). Mammalian: pCl, pSI (Promega). However, any other plasmid or vector may be used as long as they are replicable and viable in the host.

In a gene therapy approach the CDs of the present invention can be overexpressed in tumour cells by placing the gene coding for said CD under the control of a strong constitutive or tissue specific promoter, such as the CMV promoter, human UbiC promoter, JeT promoter (U.S. Pat. No. 6,555,674), SV40 promoter, and Elongation Factor 1 alpha promoter (EF1-alpha). Another type of preferred promoters include tissue specific promoters, which preferably encompass promoters that are expressed specifically in cancer cells (e.g. the intermediate filament protein nestin promoter promotes cell-specific expression in neuro-epithelial cells of stem cell or malignant phenotype (Lothian, C. et al., 1999, Identification of both general and region-specific embryonic CNS enhancer elements in the nestin promote, Exp. Cell Res., 248:509-519). Other suitable examples of tissue specific promoters include: PSA prostate specific antigen (prostate cancer); AFP Alpha-Fetoprotein (hepatocellular carcinoma); CEA Carcinoembrionic antigen (epithelial cancers); COX-2 Cyclo-oxygenase 2 (tumour); MUC1 Mucin-like glycoprotein (carcinoma cells); E2F-1 E2F transcription factor 1 (tumour). Human telomerase reverse transcriptase (hTERT), the catalytic subunit of telomerase functions to stabilise telomere length during chromosomal replication. Previous studies have shown that hTERT promoter is highly active in most tumour tissue and immortal cell lines, but inactive in normal somatic cell types.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trod, the phage lambda PL promoter and other promoters known to control expression of genes in prokaryotic or eukaryotic cells or their viruses.

Promoter regions can be selected from any desired gene using CAT (chloramphenicol transferase) vectors or other vectors with selectable markers. Two appropriate vectors are PKK232-8 and PCM7. Particular named bacterial promoters include lacI, lacZ, T3, T7, gpt, lambda PR, PL and trp.

Eukaryotic promoters include E1A (immediate early), HSV thymidine kinase, early and late SV40, LTRs from retrovirus, and mouse metallothionein-I. Selection of the appropriate vector and promoter is well within the level of ordinary skill in the art.

The constructs in host cells can be used in a conventional manner to produce the gene product encoded by the recombinant sequence. Alternatively, the polypeptides of the invention can be synthetically produced by conventional peptide synthesizers.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The expression vector also contains a ribosome binding site for translation initiation and a transcription terminator.

The vector may also include appropriate sequences for amplifying expression.

Proteins can be expressed in mammalian cells, yeast, bacteria, or other cells under the control of appropriate promoters. Cell-free translation systems can also be employed to produce such proteins using RNAs derived from the DNA constructs of the present invention.

Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Transcription of the DNA encoding the polypeptides of the present invention by higher eukaryotes may be increased by inserting an enhancer sequence into the vector. Enhancers are cis-acting elements of DNA, usually about from 10 to 300 bp that act on a promoter to increase its transcription.

Examples including the SV40 enhancer on the late side of the replication origin bp 100 to 270, a cytomegalovirus early promoter enhancer, the polyoma enhancer on the late side of the replication origin, and adenovirus enhancers.

Optionally, the heterologous sequence can encode a fusion protein including an N-terminal identification peptide imparting desired characteristics, e.g., stabilization or simplified purification of expressed recombinant product.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

### Host Cells

As representative examples of appropriate hosts, there may be mentioned: bacterial cells, such as E. coli, Bacillus subtilis, Streptomyces, Salmonella typhimurium, Pseudomonas species, Staphylococcus sp.; fungal cells, such as yeast; insect cells such as Drosophilia S2 and Spodoptera Sf9; animal cells such as CHO, COS or Bowes melanoma; adenovirus; plant cells, etc. The selection of an appropriate host is deemed to be within the scope of those skilled in the art from the teachings herein.

In a preferred embodiment the host cell of the invention is a eukaryotic cell, in particular a mammalian cell, a human cell, an oocyte, or a yeast cell. In a more preferred embodiment the host cell of the invention is a human cell, a dog cell, a monkey cell, a rat cell or a mouse cell.

The human cells may be human stem cells or human precursor cells, such as human neuronal stem cells, and human hematopoietic stem cells etc capable of forming tight junctions with cancer cells. These may be regarded as therapeutic cell lines and can be administered to a subject in need thereof. Stem cells have the advantage that they can migrate in the body and form tight junctions with cancer cells. Upon administration of 5-FC, this is converted into a cytotoxic 5-FU by the stem cell cytosine deaminase and the stem cell is killed selectively together with cancer cells. Non-limiting examples of committed precursor cells include hematopoietic cells, which are pluripotent for various blood cells; hepatocyte progenitors, which are pluripotent for bile duct epithelial cells and hepatocytes; and mesenchymal stem cells. Another example is neural restricted cells, which can generate glial cell precursors that progress to oligodendrocytes and astrocytes, and neuronal precursors that progress to neurons.

Migrating cells that are capable of tracking down glioma cells and that have been engineered to deliver a therapeutic molecule represent an ideal solution to the problem of glioma cells invading normal brain tissue. It has been demonstrated that the migratory capacity of neural stem cells (NSCs) is ideally suited to therapy in neurodegenerative disease models that require brain-wide cell replacement and gene expression. It was hypothesized that NSCs may specifically home to sites of disease within the brain. Studies have also yielded the intriguing observation that transplanted NSCs are able to home into a primary tumor mass when injected at a distance from the tumor itself; furthermore, NSCs were observed to distribute themselves throughout the tumor bed, even migrating in juxtaposition to advancing single tumor cells (Dunn & Black, Neurosurgery 2003, 52:1411-1424; Aboody et al, PNAS, 2000, 97:12846-12851). These authors showed that NSCs were capable of tracking infiltrating glioma cells in the brain tissue peripheral to the tumor mass, and “piggy back” single tumor cells to make cell-to-cell-contact.

Preferably the kind of stem cell used for this type of therapy originates from the same tissue as the tumour cell or from the same growth layer. Alternatively, the stem cells may originate from bone marrow. The stem cells may be isolated from the patient (e.g. bone marrow stem cells), be engineered to over-express a cytosine deaminase and be used in the same patient (autograft). For use in the CNS, where graft-host incompatibility does not constitute a significant problem, the cells may originate from a donor (allograft). The donor approach is preferred for the CNS as this makes it possible to produce large quantities of well-characterised stem cells, which can be stored and are ready for use. It is also contemplated to use xenografts, i.e. stem cells originating from another species, such as other primates or pigs. Cells for xenotransplantation may be engineered to reduce the risk of tissue rejection.

Salmonella typhimurium genetically modified to express the CD of the invention may also be used as a delivery vehicle for delivering the CD to cancer cells (Cunningham et al, 2001, Hum Gene Ther, 12(12):1594-6).

Bone marrow transplantation is more and more adopted as a therapy for a number of malignant and non-malignant haematological diseases, including leukemia, lymphoma, aplastic anemia, thalassemia major and immunodeficiency diseases in general. Since donor marrow contains immunocompetent cells, the graft rejects the host (causing so called graft-versus-host disease, GVHD) in 50-70% of the transplant patients, resulting in generalised inflammatory erythrodema of the skin, gastrointestinal haemorrhage and liver failure. Over 90% of GVHD cases are fatal. Although various treatments are administered to prevent GVHD in bone marrow transplantation there is clear need for safety mechanisms, which can be activated on demand to kill transplanted cells. By incorporating a CD gene of the present invention into donor cells prior to transplantation, these cells are rendered susceptible to nucleoside analogues. Nucleoside analogues can be administered in case of GVHD to stop deadly GVHD. This “safety switch” can be refined further by placing the introduced cytosine deaminase under the control of a strong inducible promoter, e.g. Tet on-off.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE Dextran mediated transfection, or electroporation. (Davis, L., Dibner, M., Battey, I., Basic Methods in Molecular Biology, (1986)).

### Recombinant Production of CDs

Following transformation or transduction of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art. Once example is expression and purification of a GST-tagged CD. The GST tag may be cleaved from the CD.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines.

The CD polypeptides may be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography hydroxylapatite chromatography and lectin chromatography. Protein refolding steps can be used, as necessary, in completing configuration of the protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated.

### Gene Therapy

The Dekkera/Brettanomyces CD polypeptides, may also be employed in accordance with the present invention by expression of such polypeptides in vivo, which is often referred to as “gene therapy.”

Thus, for example, cells from a patient may be engineered with a polynucleotide (DNA or RNA) encoding a polypeptide ex vivo, with the engineered cells then being provided to a patient to be treated with the polypeptide.

Such methods are well-known in the art. For example, cells may be engineered by procedures known in the art by use of a retroviral particle containing RNA encoding a polypeptide of the present invention. For example, the expression vehicle for engineering cells may be other than a retrovirus, for example, an adenovirus which may be used to engineer cells in vivo after combination with a suitable delivery vehicle. Most preferable is oncolytic adenovirus (replication competent adenovirus) and adenovirus. AAV and lentivirus are also preferred for some cancer applications as both types of vectors have been tested in clinical trials. Other preferred viruses include: a recombinant measles virus vector (MV), Sendai Virus Vectors (SeV), and pseudo-type Simian Immunodeficiency Virus (SIV) vector.

In U.S. Pat. No. 6,627,442 methods and viruses for efficient transduction of primary hematopoietic cells and hematopoietic stem cells are described.

Similarly, cells may be engineered in vivo for expression of a polypeptide in vivo by, for example, procedures known in the art and described in the present application.

Alternatively and for prolonged delivery of virus particles, a producer cell for producing a retroviral particle containing RNA encoding the polypeptide of the present invention may be administered to a patient for engineering cells in vivo and expression of the polypeptide in vivo.

These and other methods for administering a polypeptide of the present invention by such method should be apparent to those skilled in the art from the teachings of the present invention.

Once the Dekkera/Brettanomyces CD polypeptides are being expressed intracellularly via gene therapy, they may be employed to treat malignancies, e.g., tumors, cancer, leukemias and lymphomas and viral infections, since Dekkera/Brettanomyces CD can catalyse the conversion of 5-FC to 5-FU.

Guidance to the dosage of Dekkera/Brettanomyces CD protein, Dekkera/Brettanomyces CD virus and 5-FC can be found in the publications describing clinical trials with cytosine deaminase suicide gene therapy. Some of the cited references include the use of double genes expressing both cytosine deaminase and a thymidine kinase. (Freytag S O, et al. “Phase I study of replication-competent adenovirus-mediated double-suicide gene therapy in combination with conventional-dose three-dimensional conformal radiation therapy for the treatment of newly diagnosed, intermediate- to high-risk prostate cancer”. Cancer Res. 2003 Nov. 1; 63(21):7497-506; Freytag S O, et al. “Phase I study of replication-competent adenovirus-mediated double suicide gene therapy for the treatment of locally recurrent prostate cancer.” Cancer Res. 2002 Sep. 1; 62(17):4968-76; Cunningham C, et al. “A phase I trial of genetically modified Salmonella typhimurium expressing cytosine deaminase (TAPET-CD, VNP20029) administered by intratumoral injection in combination with 5-fluorocytosine for patients with advanced or metastatic cancer.” Hum Gene Ther. 2001 Aug. 10; 12(12):1594-6; Pandha H S, et al. “Genetic prodrug activation therapy for breast cancer: A phase I clinical trial of erbB-2-directed suicide gene expression.” J Clin Oncol. 1999 July; 17(7):2180-9; Crystal R G, et al. “Phase I study of direct administration of a replication deficient adenovirus vector containing the E. coli cytosine deaminase gene to metastatic colon carcinoma of the liver in association with the oral administration of the pro-drug 5-fluorocytosine.” Hum Gene Ther. 1997 May 20; 8(8):985-1001).

Cytosine deaminases have been used for treating the following types of cancer (see citations above), which are amenable to suicide gene therapy according to the present invention: Prostate cancer, metastatic cancer, breast cancer, colon carcinoma.

Adaptation of the dosages described in the above identified publications to the Dekkera/Brettanomyces CD described in the present application are within the capabilities of the person skilled in the art.

The CDs of the invention may be used as a “safety switch” in donor cells prior to transplantation into the host to make it possible to selectively kill the transplanted cells in the case of GVHD or in other cases, where there is a need to remove transplanted cells.

### Pharmaceutical Compositions

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

The pharmaceutical compositions may be administered in a convenient manner such as by the oral, topical, intravenous, intraperitoneal, intramuscular, subcutaneous, intranasal or intradermal routes. The pharmaceutical compositions are administered in an amount which is effective for treating and/or prophylaxis of the specific indication.

### Antibodies

The polypeptides, their fragments or other derivatives, or analogs thereof, or cells expressing them can be used as an immunogen to produce antibodies thereto. These antibodies can be, for example, polyclonal or monoclonal antibodies.

The present invention also includes chimeric, single chain, and humanized antibodies, as well as F_{ab} fragments, or the product of a F_{ab} expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBVhybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

### Industrial Scale Deamination of Cytosine Derivatives

In one aspect the invention relates to a method of deaminating a cytosine derivative, comprising exposing said cytosine derivative to a cytosine deaminase according to the invention and recovering the deaminated cytosine derivative.

Cytosine deaminases according to the present invention combine a higher conversion rate of synthetic analogs (cytosine derivatives) exemplified by 5-FC with higher thermostability thus providing improvements in the industrial scale deamination of such cytosine derivatives.

The cytosine derivative may be is selected from the group consisting of 2-thiocytosine, 6-aza-cytosine, 4-aza-cytosine and 5-FC. The invention also relates to a method of producing 5-fluorouracil comprising subjecting 5-fluorocytosine to a cytosine deaminase according to the invention and recovering the 5-FU.

The process may be carried out at a temperature above 35° C., preferably above 370, more preferably above 40° C., more preferably above 45° C., more preferably above 50° C.

### Use of 5-Fluorocytosine for Controlling the Growth of Dekkera/Brettanomyces.

In one aspect the invention relates to the use of 5-fluorocytosine for controlling the growth of Dekkera/Brettanomyces yeast. The yeast of Brettanomyces are well-known wine spoilage yeast which produce off-flavours such as volative phenols, acetic acid and tetrahydropyridines. Although these yeasts are not normally found on grapes and in fermenting must, they can develop at the end of the alcoholic fermentation and durine wine ageing in wooden barrels. This is in part caused by the ability of these yeasts to grow at high ethanol concentrations and at low pH. These yeast may similarly spoil beer and other fermented alcoholic beverages. There are some basic methods for prevention of Brettanomyces growth in wine, but most have detrimental effects on wine quality. Decreasing pH, increasing SO₂, decreasing aging temperature, avoiding barrels, and sterile filtration are all effective at controlling Brettanomyces, yet they pose obvious problems to winemakers.

As described in the appended examples, all tested strains of Dekkera/Brettanomyces are particularly susceptible to 5-fluorocytosine. Therefore 5-fluorcytosine can be used to control the growth of these yeasts. Dekkera/Brettanomyces can represent a problem to all kinds of fermented alcoholic beverages that are subject to ageing and/or storage, in particular when the beverage is aged and/or stored in direct connection with wood. As the yeasts are of particular damage to wine and beer, 5-fluorocytosine is preferably used during wine or beer making and/or ageing and/or storage. One component of wine off flavour is represented by 4-ethyl-phenol. A useful sensory threshold to use for 4-ethyl-phenol is 420 micrograms/litre. At this concentration and beyond, a wine will typically be noticeably bretty. Below this concentration, the character of the wine may be changed but people will not, on average, recognize that this is due to 4-ethyl-phenol.

5-FC may be added to the wine or beer before or during ageing or storage. 5-FC is approved as a drug for human beings and is non-toxic to humans as mammals are not able to convert the compound into the cytotoxic product, 5-FU. Therefore, the presence of small amounts of 5-FC in beer, wine or other fermented alcoholic beverages should not represent a health problem. Furthermore, 5-FC is quickly degraded in the presence of light and will thus normally be eliminated from the beverage before consumption.

The majority of wines became infected by these yeasts during the period of barrel maturation, particularly if second use (or older) oak barrels were used. Brettanomyces can colonise a barrel between fills, and can begin to reproduce when the barrel is refilled with new wine. However, the use of new barrels does not guarantee that these yeasts will not appear. Even new barrels filled with sterilized wine can still sustain populations of Brettanomyces yeasts high enough to produce above threshold levels of 4-ep which results in sensory modification to the wine. Wood is virtually impossible to sanitize and since Brettanomyces produce the enzyme β-glucosidase which allows it to grow on the wood sugar cellobiose. New barrels contain higher amounts of cellobiose than used barrels, and therefore have the potential to support higher Brettanomyces populations. Cellobiose in barrels occurs as a result of the firing process wine makers use to toast the barrels. The β-glucosidase enzyme of Brettanomyces cleaves the disaccharide cellobiose to produce glucose molecules which are then used for growth.

The 5-FC may additionally or alternatively be applied to the outside or inside of containers and/or to utensils before or during making and/or ageing and/or storage. For example wooden barrels may be soaked in an aqueous solution of 5-FC prior to usage for ageing of the fermented alcoholic beverage. In some cases the beverage is stored in stainless steel tanks and wood is added as wood chunks. Such wood chunks may also preferably be soaked in an aqueous solution of 5-FC prior to contact with the fermented alcoholic beverage.

Preferably the concentration of 5-FC is below 1 μM, more preferably below 0.5 μM, more preferably below 0.36 μM, more preferably below 0.1 μM.

### EXAMPLES

### Example 1

### Cloning and Characterisation of a Novel Cytosine Deaminase Gene from D. bruxellensis

### Material and Methods

### Chemicals

TOPO TA Cloning® kit, pET vectors, isopropyl-1-thio-b-D-galactopyranoside (IPTG), DNA and protein molecular weight standards were from Invitrogen. Unlabeled nucleobases, 5-fluorocytosine and 5-fluorouracil were from Sigma. Radioactively labelled nucleobases were obtained from Moravek Biochemicals Inc. (Brea, Calif.). Unless specified otherwise all cell culturing media, serum and gentamicin were from Cambrex, Bio Whittaker (Belgium).

### Strains and Growth Media

The yeast strains used in this work are: Dekkera bruxellensis (Y872, CBS 1943), D. bruxellensis (Y879, CBS 2499), D. bruxellensis (CBS 4480, CBS 4481), D. anomala (CBS 76, CBS 77, CBS 1938, CBS 1947), Brettanomyces nanus (CBS 1945), B. nanus (CBS 1955, CBS 1956), M. reukauffii (CBS 2266), B. custersianus (CBS 4805), B. naardensis (CBS 6042), S. kluyveri Y057 (NRRL Y-12651), and S. cerevisiae Y051 (NRRL Y-12632). Yeast strains were grown at 25° in YPD medium (1% yeast extract, 2% bacto peptone, 2% glucose) or in defined minimal (SD) medium (1% succinic acid, 0.6% NaOH, 2% glucose, 0.67% yeast nitrogen base without amino acids from Difco). When indicated, (NH₄)₂SO₄ was replaced with 0.1% cytosine as the sole nitrogen source giving N-minimal medium. The growth rate was determined in liquid medium by following the optical density at 600 nm. The E. coli strain TOP10 (Invitrogen) was used for plasmid amplification. Bacteria were grown at 370 in Luria-Bertani medium supplemented with 100 mg/l of ampicillin for selection. The E. coli BL21-DE3 (Invitrogen) strain was used for heterologous protein expression.

### DNA and RNA Isolation

Yeast genomic DNA was isolated using zymolyase and standard procedures (Johnston, J. R., Molecular Genetics of Yeast: A Practical Approach, IRL Press/Oxford University Press, Oxford (1994)). Nucleospin Blood Quick Pure kit (Macherey-Nagel). Total RNA was isolated from yeast cells grown in N-minimal cytosine medium using FastRNA Red kit (Bio101) and FastPrep machine FP120 (Bio101 Savant) according to supplier's directions. Integrity of RNA was analyzed by RNA6000 Nano Chips on Agilent 2100 Bioanalyzer. RT-PCR was performed using SuperScript™ One-Step RT-PCR Systems (Invitrogen).

### Degenerative PCR and Genome Walking

Degenerative primers were made using the BLOCKS- and the CODEHOP-webinterface (Fred Hutchinson Cancer Research Center) Rose, T. M., Schultz, E. R., Henikoff, J. G., Pietrokovski, S., McCallum, C. M., and Henikoff, S. (1998) Nucleic Acids Res. 26, 1628-1635). Based on the FASTA-file, containing peptide sequences of CD from different ascomycetous yeasts, the BLOCKS computed three conserved regions which were submitted to the CODEHOP web server using the standard settings and S. cerevisiae genetic code. Chosen primers were:

P425-5′ TGCAAAAGGTTATAAAGAAGGTGGTRTNCCNATHGG 3′
P427-5′ CTTGGAATACCATACATTATAATAGCACCNGYRCACAT 3′
P428-5′ CTGAGAATTCCTATTCACCAATATCTTCRWWCCARTC 3′

Upstream and downstream sequences of CD gene were obtained using DNA Walking SpeedUp™ kit (Seegene Inc., Seoul, Korea).

### Cloning and Analysis of CD Genes

FCY1 gene from S. cerevisiae (Acc. nr. U55193) was obtained from genomic DNA by PCR amplification using Accuzyme DNA polymerase (Bioline). cDNA for D. bruxellensis CD gene was obtained by RT-PCR using total RNA. The PCR products were directly ligated into pET100 and pET101 vectors (Invitrogene) allowing expression of the protein encoded by the open reading frame fused to the histidine tag. The sequences of the expression inserts were verified by sequencing and designated as PZG738 (Sc CD-pET100) and PZG______ (Db CD-pET100). CD sequences were aligned using the ClustalX 1.81 program (Jeanmougin, F., Thompson, J. D., Gouy, M., Higgins, D. G., and Gibson, T. J. (1998) Trends Biochem. Sci. 23, 403-405), and a phylogenetic analysis was performed {Van de Peer, Y. and De Watcher, R., (1994) Comput. Appl. Biosci. 10, 569-570).

### Promoter Studies

For the promoter studies app. 1.1 kb of CD promoters were cloned into EcoRI/BamHI site of pYLZ-2 plasmid (Hermann, H., Hacker, U., Bandlow, W., and Magdolen, V. (1992) Gene 119, 137-141), leading to fusion to lacZ gene (PZG875 and PZG877). Two constructs, PZG877 (CD1 promoter, from −1075 to start codon) and PZG875 (CD2 promoter, from −1102 to start codon) were made. β-galactosidase assay (Sambrook, J. and Russell, D. W., Molecular Cloning: A Laboratory Manual, Third Edition, Cold Spring Harbor, N.Y., (2001)) using ONPG as a substrate was performed on S. cerevisiae cells transformed with the plasmids. Cells were opened with glass beads using FastPrep machine (maximal speed, 30 sec.) in 0.1 M sodium phosphate buffer (pH 7.5) and assayed immediately.

### Purification of the Recombinant Enzymes

For recombinant protein expression, E. coli cells were grown to a density of A_{600 nm}=0.5-0.6 in LB medium supplemented with 100 μg/ml ampicillin, and the expression was induced with 10 μM IPTG for 8 h at 25° C. The cells were harvested by centrifugation, and the pellet was resuspended in 25 ml ice-cold binding buffer A (50 mM Tris HCL pH 7.5, 1 mM DTT, 10% Glycerol, 1% TritonX-100) (50 mM sodium phosphate pH 8.0; 300 mM NaCl; 10% glycerol; 25 mM imidazole) containing protease inhibitor cocktail (Complete™-EDTA free from Roche Diagnostics). The cells were homogenized using a French Press, subjected to centrifugation at 12,000×g for 30 minutes (4° C.), filtered through a 1 mm Whatman glass microfiber filter and a 0.45 mm cellulose acetate filter, and loaded onto a 5 ml Ni²⁺-NTA column (Qiagen). The column was washed with 10 vol of buffer A, 10 vol of buffer B (50 mM sodium phosphate pH 6.0; 300 mM NaCl; 10% glycerol; 25 mM imidazole) and finally with 10 vol of buffer B containing 50 mM imidiazole. The recombinant CD was eluted from the column by a linear gradient of 50 to 500 mM imidazole in buffer B. Fractions containing recombinant protein were precipitated by ammonium sulfate (70% saturation at 0°), resuspended in Tris buffer (50 mM Tris HCl pH 7.5, 100 mM NaCl, 1 mM DTT), and than applied to G-25 column and stored at −80° at a concentration of 10 mg/ml.

SDS-PAGE was done as described by Laemmli (Laemmli, U. K. (1970) Nature 227, 680-685) and proteins were visualized by SimplyBlue™ Safestain (Invitrogen). The protein concentration was determined using Bio-Rad Protein Assay (Bio-Rad) and BSA as standard protein (Bradford, M. M. (1976) Anal. Biochem. 72, 248-254). Alternatively proteins were analyzed on Agilent 2100 Bioanalyzer using Protein Assay Chips.

### Enzyme Assays

Bacteria or cell lines were grown as described, harvested and stored at −80° C. until activity testing. Cells were submitted to brief sonication in extraction buffer (50 mM Tris/HCl pH 7.5, 1 mM DTT, 10% (v/v) glycerol, 1% (v/v) Triton X-100, protease inhibitor cocktail Complete™ from Roche Diagnostics). Cytosine deaminase activities were determined spectrophotometrically by monitoring the absorbance decrease at 286 nm (Δε₂₈₆=−0.68 mM⁻¹ cm⁻¹) resulting from the deamination of cytosine to uracil. Assays were initiated by the addition of CD to 0.5 mM cytosine in 50 mM Tris-HCL at pH 7.5 and 30° C. and followed for 150 sec. One unit of CD was defined to catalyze the formation of 1 μmol of uracil per min. Dual beam recording ZEIS spectrophotometer, thermostated at 30° C., was used for the assays.

### Plate Screening of CD Genes

The TOP10 E. coli strain was transformed by heat shock with the expression plasmids using standard techniques and plated on LB-ampicillin (100 mg/ml) plates containing 10 μM IPTG. Selection of mutants was done on M9 minimal medium plates (Ausubel, F.; Brent, R.; Kingston, R. E.; Moore, D. D.; Seidman, J. G.; Smith, J. A.; Struhl, K., Short Protocols in Molecular Biology, 3^{rd}. edition Wiley, New York. (1995)) containing different concentrations of 5-FC. Plates were prepared by mixing the medium at 56° C. with the 5-FC, before pouring the plates. Growth of colonies was visually inspected after 24 hours at 37° C. From clones not growing on analog-containing plates, but growing normally on control plates, the plasmid was isolated and retransformed into TOP10. These clones were retested to verify the plasmid-borne phenotype. All clones with increased sensitivity towards 5-FC were tested again on plates with logarithmic dilutions of the analog to determine the lethal doses (LD₁₀₀) of the 5-FC, at which no growth of bacteria could be seen.

### Results

Dekkera bruxellensis Catabolizes Cytosine

The majority of yeast can utilize cytosine as sole source of nitrogen by using CD to cleave cytosine to ammonia (a source of nitrogen) and uracil. To test for the ability of Dekkera yeast for growth on cytosine, Y872 was spotted on N-minimal media containing 0.1% cytosine. After 5 days growth was observed although it was slower compared to the growth on YPD or SD medium (FIG. 1). Since these results indicated that Dekkera yeasts have both functional uptake and deamination of cytosine, sensitivity towards 5-FC was tested. Dekkera yeasts were very sensitive to 5-FC. On SD medium addition of 0.1 μM of 5-FC highly suppressed growth, while on plate containing 0.36 μM of 5-FC no growth was seen. (FIG. 1). At the same time S. cerevisiae growth was only inhibited at plates containing 1 μM of 5-FC. Therefore it seems that D. bruxellensis CD may have better substrate specificity towards 5-FC and converts more 5-FC into 5-FU which ultimately leads to the stronger cell killing.

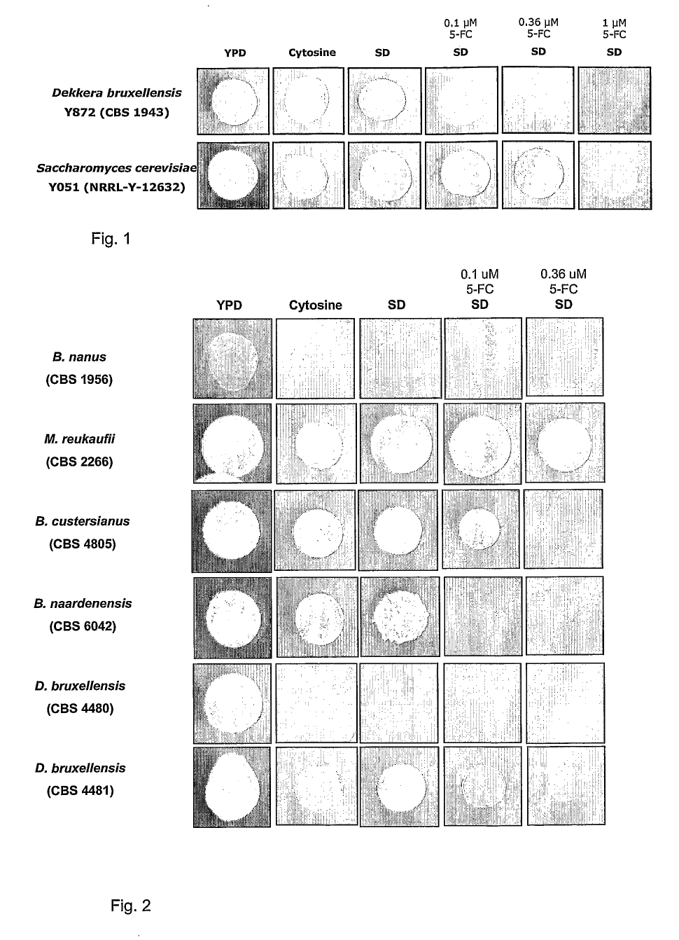

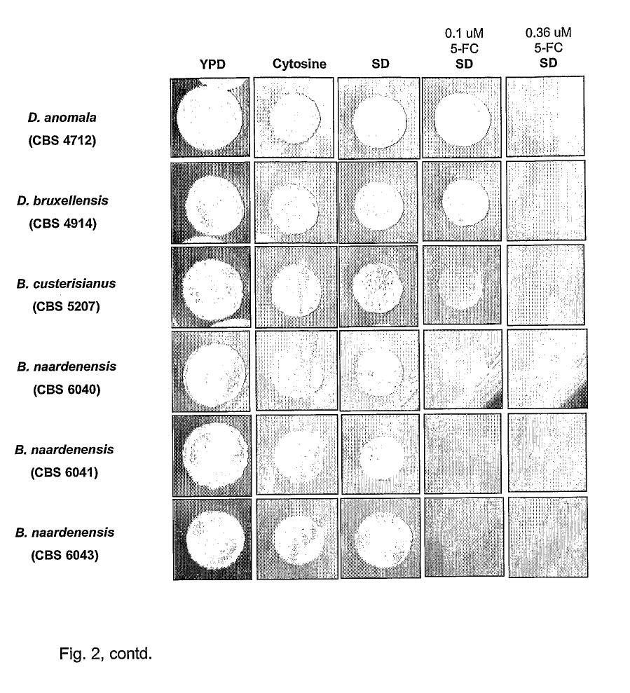

In FIG. 2 it can be seen that all the tested Dekkera/Brettanomyces strains are particularly susceptible to 5-FC, as growth is inhibited at 0.1 or at least at 0.36 μM 5.FC. For comparison, S. cerevisiae (FIG. 1) and M. reukauffii (FIG. 2) strains are included.

D. bruxellensis CD has Unique Gene Organization

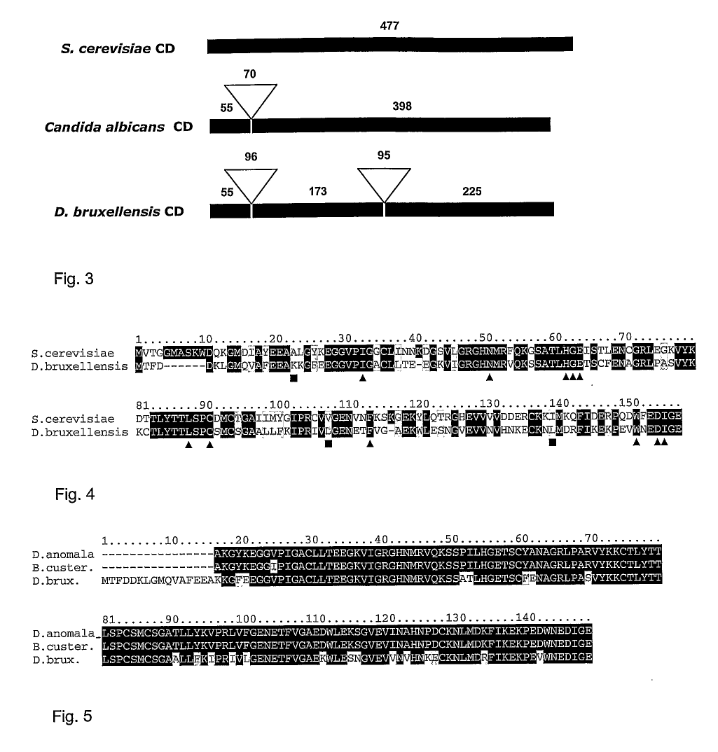

Having shown that Dekkera bruxellesis can utilize cytosine as sole nitrogen source and has a functional CD gene we attempted to clone CD from total genomic DNA using degenerative primers based on multiple alignment of CDs from 18 ascomycetous yeasts. Using P425 and P427 primers, δ 250 bp long PCR fragment was expected to be amplified. However, a PCR fragment of 350 bp was obtained and subsequently cloned into TOPO TA cloning vector. Translation of the sequenced fragment revealed homology to the S. cerevisiae CD gene but the middle part contained several stop codons and could not be translated in the right frame. A putative intron of 95 bp was predicted and its presence was confirmed by sequencing of Y872 cDNA. The position of this intron is quite unique; Saccharomyces yeasts have intronless CD while Candida albicans CD gene has intron at the beginning of the gene. Having determined the partial sequence of D. bruxellensis CD gene, upstream and downstream sequence was obtained using gene walking. A total of 1804 bp were obtained. One additional intron was predicted in the middle of the gene. So far D. bruxellensis CD gene is the only one containing two introns (FIG. 3). Therefore this gene organization is quite unique among fungal CDs. The isolated cDNA codes for an ORF of 453 bp (SEQ ID NO 1) encoding a protein of 150 amino acid (aa) residues. The calculated molecular mass of the D. bruxellensis CD protein was 16547 Da with 5.5 μl. The greatest similarity of the protein was to the putative CDs from Debaryomyces hansenii (64% identities), Aspergilus fumigatus (63% identities) and Candida albicans (62% identities). The gene was named Db CD1.

Multiple alignment of protein sequences showed that all amino acid residues from S. cerevisiae CD involved in the binding of metal ion and pyrimidine ring (Ireton, G. C., Black, M. E., and Stoddard, B. L. (2003) Structure. (Camb.) 11, 961-972; Sabini, E., Ort, S., Monnerjahn, C., Konrad, M., and Lavie, A. (2003) Nat. Struct. Biol. 10, 513-519) are conserved in D. bruxellensis CD (FIG. 4). In addition residues of the S. cerevisiae CD which can lead to higher termostability of the enzyme (Korkegian, A., Black, M. E., Baker, D., Stoddard, B. L., (2005) Science 308, 857-860.) were not conserved and it seems that D. bruxellensis wild type enzyme already contains several residues which may give a termostable enzyme (FIG. 4).

### Promoter Activity

During attempts to clone 5′ part of CD gene several PCR fragments from gene walking experiments were obtained. Fragments larger than 1 kb were sequenced and surprisingly two different sequences with app. 90% identity to each other were found. The regions upstream of the start codon were subcloned into high-copy pYLZ-2 plasmid containing the lacZ gene as a reporter. The plasmids were transformed into S. cerevisiae and independent transformants were grown in various media (Table 1). β-galactosidase activity was observed in cells grown on YPD and SD media, but much higher activity of the reporter gene was found in cells grown on cytosine as the only source of nitrogen. CD2 promoter had only one-half of activity compared to the CD1 promoter in all media tested.

TABLE 1
β-galactosidase assays containing CD1
and CD2 promoters fused to the lacZ gene.

| | β-galactosidase activity (nmol/mg/min) | | |
|---|---|---|---|
| Medium | CD1p | CD2p | pYLZ-2 |
| YPD | 4.42 ± 0.13 | 2.47 ± 0.22 | 0.18 ± 0.13 |
| SD | 5.62 ± 0.77 | 2.11 ± 0.14 | 0.04 ± 0.04 |
| Cytosine | 23.22 ± 1.16 | 9.43 ± 0.11 | 0.05 ± 0.05 |

The obtained results indicated that CD1 gene might be the major enzyme contributing to the conversion of cytosine into uracil in D. bruxellensis. Therefore we decided to study this gene in more details.

### Expression of Yeast CDs

To better characterize D. bruxellensis CD over expression of the protein in bacteria was done. In addition to D. bruxellensis CD, homologous genes from S. cerevisiae was also amplified from total genomic DNA and cloned as histidin tagged construct into pET100 bacterial expression vector. The plasmids were transformed into E. coli BL21 strain and protein expression was induced by IPTG for 8 hours. After harvesting, cell extracts were spectrophotometrically measured for cytosine and 5-FC deamination. All yeast CDs tested were functional when expressed in bacteria (Table 2).

TABLE 2
CD activities in crude extracts of BL21 cells
transformed with different CD genes.

| Transformant | Cytosine | 5-FC |
|---|---|---|
| Cells only | n.d. | |
| pET100 | n.d. | |
| PZG738 (Sc CD-pET100) | 40.69 ± 0.56 | |
| PZG__ (Db CD-pET100) | | |

The cytosine was tested at a fixed concentration of 500 uM. All assays were performed in triplicates and the results presented are the mean values with standard deviation.

The CD enzymes from different yeast are cloned in Moloney murine leukemia virus to create replication-deficient recombinant retroviridae with and without the yeast CD. Human glioblastoma cell line U87-MG and breast cancer cell line MCF-7 are transduced with the retroviridae, and stable polyclonal populations of cells created.

### Example 2

### Dekkera bruxellensis Sequences

```
Y872 D. bruxellensis CD1 gene
cDNA 453 bp
(SEQ ID NO 1)
ATGACATTTGATGACAAATTAGGAATGCAGGTTGCCTTCGAGGAGGCCAAAAAGGGATTT
GAGGAAGGAGGTGTCCCTATTGGAGCATGTCTGCTTACCGAGGAGGGAAAGGTGATTGGT
CGTGGCCACAATATGCGTGTTCAGAAGTCATCTGCCACTCTTCATGGTGAAACATCATGT
TTTGAGAATGCCGGAAGATTGCCCGCTTCTGTTTACAAGAAATGCACGCTTTACACCACT
TTGTCTCCATGCTCCATGTGCAGTGGTGCAGCCTTGTTGTTCAAGATTCCAAGGATTGTT
CTTGGAGAAAACGAGACGTTTGTTGGTGCAGAGAAGTGGCTTGAGAGTAATGGAGTGGAA
GTTGTGAATGTGCATAACAAAGAGTGCAAAAATCTCATGGATAGGTTTATTAAGGAGAAG
CCAGAGGTCTGGAATGAGGATATTGGCGAGTAA
Protein 150 aa Theoretical pI/Mw: 5.50/16546.97
(SEQ ID NO 2)
MTFDDKLGMQVAFEEAKKGFEEGGVPIGACLLTEEGKVIGRGHNMRVQKSSATLHGETSC
FENAGRLPASVYKKCTLYTTLSPCSMCSGAALLFKIPRIVLGENETFVGAEKWLESNGVE
VVNVHNKECKNLMDRFIKEKPEVWNEDIGE
(SEQ ID NO 1 and 2)
atgacatttgatgacaaattaggaatgcaggttgccttcgaggaggccaaaaagggattt
  M T F D D K L G M Q V A F E R A K K G F
gaggaaggaggtgtccctattggagcatgtctgcttaacgaggagggaaaggtgattggt
  E E G G V P I G A C L L T E E G K V I G
cgtggccacaatatgcgtgttcagaagtcatctgccactcttcatggtgaaacatcatgt
  R G H N M R V Q K S S A T L H G E T S C
tttgagaatgccggaagattgcccgcttctgtttacaagaaatgcacgctttacaccact
  F E N A G R L P A S V V K K C T L V P T
ttgtctccatgctccatgtgcagtggtgcagccttgttgttcaagattccaaggattgtt
  L S P C S M C S G A A L L F K I P R I V
cttggagaaaacgagacgtttgttggtgcagagaagtggcttgagagtaatggagtggaa
  L G E N E T F V G A E K W L E S N G V H
gttgtgaatgtgcataacaaagagtgcaaaaatctcatggataggtttattaaggagaag
  V V N V H N K E C K N L M D R F I K E K
ccagaggtctggaatgaggatattggcgagtaa
  P E V W N E D I G E -
Genomic sequence including protein sequence
(SEQ ID NO 3 and 2)
```

```
                                                                              ggggggggggtctaattgcgg
tatgctaaattcttgcactgctagttcaattgtgcaacaacgcagatattctctaagtaa
atgttttatccgaccaaaaagcccgtcaattctataatatcgacctgtggcctttctcta
gaatattctcccttcgttkatatatatttgtttcctcttcttcaactcatctcaaactga
aactcgtgcgaacaagtttaaattattattcctattgcaattcagttctgtttttttttt
actttttcgagttcgtcattcgtgccgcacataatccaaaggggtggctggatcggatct
tcctgttgatatggtgtacggatgtgctacgctttcgatgcaagcgggttggactgcatt
tgtagcagaacaagttatgcaagttaLgcaagttataatgccagtttaactaaaagttgg
cgctatacccatcaaaggcttgatgtcgttgattaacgaataaaataatatccgcaattt
gtgtgtgttaatgcatatataaatcgtgcgaaagtcatgcatcaaaattcacaaaacttg
agaaggtgagtattatctgaagattaatttaataacaattgggaattaacaatttgacga
agataaataaattgacggaaatagtttaaacgacaacgagcgaaatgcattcaaagtaaa
tagctgtaataacaattttagagccaacattgcaacatttaacaaccatcatccacatca
taaaaagctgtcacaatgcaatcaactcaattagccaactatatgttggcaaaattgcac
attttcaagaactttctggaatcgcattagctaaacaatttattctcccatgagttaatc
atacatacccttaaactagcacatgaatagatgaagtccctcgcattagtggtcacgtga
cataccgcccaacttggaaagtgcagcagataaatcgaaaaaagataaggagcgaggtga
aaattcaacggtggtaaaattttaaatttcacatttctgctatctttcggccccaatact
ttgaaaagcatctggctgaatatacaacccacacatatttagaactacagaaccgaaatt
atgacatttgatgacaaattaggaatgcaggttgccttcgaggaggccaaaaagggtagg
  M T F D D K L G M Q V A F E E A K K
ctgtggagtgtttaagtgcgaaaaatttaacttgtgccccagaataaaacagcattacta
acattaagcttgccgattccgctttttttaggatttgaggaaggaggtgtccctattgga
                                                              G F E E G G V P I G
gcatgtctgcttaccgaggagggaaaggtgattggtcgtggccacaatatgcgtgttcag
  A C L L T E E G K V I G R G H N M R V Q
aagtcatctgccactcttcatggtgaaacatcatgttttgagaatgccggaagattgccc
  K S S A T L H G E T S C F E N A G R L P
gcttctgtttacaagaaatgcacggtaagtatagtaagagatgcggaaacatgaagcatt
  A S V Y K K C T
cttgttgaggtcatgcttttgaactgtgtgtactaaccacagaaccatattcgatacagc
tttacaccactttgtctccatgctccatgtgcagtggtgcagccttgttgttcaagattc
L Y T T L S P C S M C S G A A L L F K I
caaggattgttcttggagaaaacgagacgtttgttggtgcagagaagtggcttgagagta
P R I V L G E N E T F V G A E K W L E S
atggagtggaagttgtgaatgtgcataacaaagagtgcaaaaatctcatggataggttta
N G V E V V N V H N K E C K N L M D R F
ttaaggagaagccagaggtctggaatgaggatattggcgagtaaaaagtctgcgaaagtt
I K E K P E V W N E D I G E -
tatgaaatttggcgaaaggttgtgaagttttgtcagattttgacaatatgtttgacgaaa
tttacgtaaaagagatgcatgagttggacacacaccaatttttatagaatgtactaggac
taatgctagatctgagcacttactggcgaatgagttgatgatttagacagtttgagacca
aaagaagatagttgaggaaaagcgagagtagttgaagattcgatctttatacttggtaaa
tggtaattgaaatagatcattacattgatgaagattagctaaagagtggatcattacatt
gatagagataggagtggatcattacattgatagagataggagtggatcattacattgata
gagataagagtggatcattacattgatagagataagagtggatcattacatttggcaaga
aagtatgagatcaaagacaatcatattgataaagatctaggcttgaatataaacacgtac
atccgtaaaaacctgcatcatgtctttcgtgacaacaatataccaactgccaataaagct
agaagtggagaaaaaatatacagtgtcagtagaaatacaccattggataatccatttgat
ttcactttgatttcacttcactcttactcttagcacttgatcagctttacggattactcc
agtatcaaatgggggtgccatgcgaacaataaaacagtaaaaagtagaacaaataataac
ggatgaagcccattagcagcggtaagataaggcaagcttgccgaacagtgggaaaggcag
tgaaaaaagggaagccaaaaagtcccaagaatactccaacaatgaattttttagttcggc
aaattcaccagcgcaaaaaatggaagggcaacagttagcaatgctggcgatattttcaaa
ctagtacaattagacccccccccctcgcttggcatacttctgtgaa
```

### Example 3

### Cloning of CD Genes from Other Dekkera/Brettanomyces Yeasts

Until now molecular and genetic system for Dekkera/Brettanomyces yeasts did not exist. There are no suitable auxotrophic markers or efficient transformation system to work with these yeasts. Since plasmids and promoter elements from S. cerevisiae and related yeasts do not work in Dekkera/Brettanomyces yeasts alternative methods for gene cloning are needed. Using degenerative primers described in this study it was not possible to amplify CD genes from D. anomala, B. nanus, B. naardenensis and B. custersianus probably due to presence of introns in these genes. Therefore we designed a selection system based on Dekkera bruxellensis CD1 promoter and G418 resistance marker coding for 3′aminoglycoside-phosphotransferase. G418 is a ribosomal inhibitor in many eukaryotic cells but many yeast species are not sensitive to G418. However, almost all Dekkera/Brettanomyces yeasts tested were sensitive to addition of 200 μg/ml. Since G418 cannot be used in medium with high salt concentration (like SD medium) use of CD1 promoter which had activity in YPD medium (low salt) will permit expression of G418 gene and ultimately lead to resistance to G418 and enable direct selection of yeast colonies containing this gene. Cloning strategy was as described: pYES2 vector was cut with AgeI/HindIII restriction enzymes and fragment of app. 440 bp containing GAL promoter was removed. Promoter of 1075 bp from Y872 CD1 gene was cloned into AgeI/HindIII site resulting in pDbCD1p plasmid. The ORF from G418 gene (Acc. nr. S78175) was cloned under control of CD1 promoter into BamHI/EcoRI site. Thereafter 2280 bp fragment containing CD1 promoter and G418 gene was amplified by PCR using primers TrzKanMX3Xmal 5′ tccccccgggAAGGAAGACTCTCCTCCGTGCG 3′ and TrzKanMX3SalI 5′ tatggccgacgtcgacTTGGCCGATTCATTAATGCAGGGCC 3′. Subsequently this fragment was transferred into Xmal/SalI site of pMOD-3 vector (EpiCentre, catalog nr. MOD1503)) and used for EZ-Tn5 transposon mutagenensis according to suppliers recommendations. Yeast cells were transformed by electroporation using sorbitol and plated on YPD plates containing G418 (selecting for integrated transposons) and thereafter replica-plated on SD plates containing 1 μM 5-FC (selecting for CD gene disruption). Total DNA from colonies able to grow on this medium was isolated and used to make a library of circular plasmids which were transformed into E. coli EC100D pir+ strain (EpiCentre, catalog nr. EC6P095H)). Plasmids from kanamycin resistant clones were isolated and fully sequenced. Transposon construct containing CD1 promoter and G418 gene integrated randomly into yeast genome and in some cases disrupted CD gene enabling the cells to become resistant to 5-FC. This strategy, using native promoter from Dekkera/Brettanomyces yeasts and dominant selection marker, provided for the first time method for cloning of CD genes in these yeasts.

### Example 4

### Cloning of a Cytosine Deaminase Gene from D. Anomala and B. custersianus

Having shown that Dekkera anomala and Brettanomyces custersianus also have functional metabolism for cytosine we cloned corresponding CD genes from total genomic DNA using P425 and P428 degenerative primers. PCR fragments of app. 500 bp were obtained and subsequently cloned into TOPO TA cloning vector. Sequencing revealed that we indeed cloned CD genes from these yeasts. Putative intron of 58 bp was predicted at beginning of the gene. Almost 90% of the CD gene was cloned in this way. The full sequence of the CD genes may be obtained with genome walking as described for D. bruxellensis. Alternatively, to obtain a full length sequence, the sequence coding for the missing N-terminal amino acids of D. bruxellensis can be ligated into the partial sequences of coding for D. anomala and B. custerianus CD.

The CD genes from D. anomala and B. custersianus shared 99% identity on both nucleotide and amino acid level. The genes shared 75% identities on nucleotide level with D. bruxellensis CD gene, while protein sequence exhibited 84% identities (106/125) and 90% positives (114/125). At the same time D. anomala and B. custersianus CD proteins showed 63% identities (72/113) and 77% positives (88/113) with Candida albicans CD enzyme.

D. anomala CD gene (C8S4712)
Predicted cDNA
(sequence of degenerative primers bold underlined)

(SEQ ID NO 4)

TGCAAAAGGTTATAAAGAAGGTGGTGTACCCATCGGTGCATGCTTGTTAA
CTGAGGAGGGTAAAGTTATTGGTCGTGGTCACAATATGAGGGTGCAGAAG
TCTTCACCAATTCTTCACGGAGAAACTTCTTGCTACGCCAATGCAGGAAG
ATTACCTGCTCGTGTTTACAAGAAATGTACTCTTTACACCACCTTGTCTC
CATGCTCTATGTGTAGTGGTGCTACACTACTTTATAAAGTTCCAAGGCTC
GTTTTCGGTGAAAATGAAACTTTTGTTGGTGCCGAGGATTGGTTAGAAAA
GAGTGGTGTTGAAGTTATCAACGCTCACAATCCTGACTGTAAGAATTTGA
TGGATAAGTTCATCAAGGAGAAGCCAGAAGACTGGAATGAAGATATTGGT
GAATAG
Translated PrOtein

(SEQ ID NO. 5)

AKGYKEGGVPIGACLLTEEGKVIGRGKNMRVQKSSPILHGETSCYANAGR
LPARVYKKCTLYTTLSPCSMCSGATLLYKVPRLVFGENETFVGAEDWLEK
SGVEVINAHNPDCKNLMDKFIKEKPEDWNEDIGE
Genomic sequence
(predicted intron and stop codon are in bold)

(SEQ ID NO 6)

TGCAAAAGGTTATAAAGAAGGTGGTGTACCCATCGGTAAGTATAATTACT
ATGATTTATTGCTAAACAAATCATTACTAACTGTCATTATTAGGTGCATG
CTTGTTAACTGAGGAGGGTAAAGTTATTGGTCGTGGTCACAATATGAGGG
TGCAGAGTCTTCACCAATTCTTCACGGAGAAACTTGCTTGCTACGCCAAT
GCAGGAAGATTACCTGCTCGTGTTTACAAGAAATGTACTCTTTACACCAC
CTTGTCTCCATGCTCTATGTGTAGTGGTGCTACACTACTTTATAAAGTTC
CAAGGCTCGTTTTCGGTGAAAATGAAACTTTTGTTGGTGCCGAGGATTGG
TTAGAAAAGAGTGGTGTTGAAGTTATCAACGCTCACAATCCTGACTGTAA
GAATTTGATGGATAAGTTCATCAAGGAGAAGCCAGAAGACTGGAATGAAG
ATATTGGTGAATAGGAATTCTCAGA
B. custersianus CD (CBS 4805)
Predicted cDNA
(sequence of degenerative primers bold underlined)

(SEQ ID NO 7)

TGCAAAAGGTTATAAAGAAGGTGGTATACCCATCGGTGCATGCTTGTTAA
CTGAGGAGGGTAAAGTTATTGGTCGTGGTCACAATATGAGGGTGCAGAAG
TCTTCACCAATTCTTCACGGAGAAACTTCTTGCTACGCCAATGCAGGAAG
ATTACCTGCTCGTGTTTACAAGAAATGTACTCTTTACACCACCTTGTCTC
CATGCTCTATGTGTAGTGGTGCTACACTACTTTATAAAGTTCCAAGGCTC
GTTTTCGGTGAAAATGAAACTTTTGTTGGTGCCGAGGATTGGTTAGAAAA
GAGTGGTGTTGAAGTTATCAACGCTCACAATCCTGACTGTAAGAATTTGA
TGGATAAGTTCATCAAGGAGAAGCCAGAAGACTGGAACGAAGATATTGGT
GAATAG
Translated Protein

(SEQ ID NO 8)

AKGYKEGGIPIGACLLTEEGKVIGRGHNMRVQKSSPILHGETSCYANAGR
LPARVYKKCTLYTTLSPCSMCSGATLLYKVPRLVFGENETFVGAEDWLEK
SGVEVINAHNPDCKNLMDKFIKEKPEDWNEDIGE
Genomic sequence
(predicted intron and stop codon are in bold)

(SEQ ID NO 9)

TGCAAAAGGTTATAAAGAAGGTGGTATACCCATCGGTAAGTATAATTACT
ATAATTTATTGCTAAACAAATCATTACTAACTGTCATTATTAGGTGCATG
CTTGTTAACTGAGGAGGGTAAAGTTATTGGTCGTGGTCACAATATGAGGG
TGCAGAAGTCTTCACCAATTCTTCACGGAGAAACTTCTTGCTACGCCAAT
GCAGGAAGATTACCTGCTCGTGTTTACAAGAAATGTACTCTTTACACCAC
CTTGTCTCCATGCTCTATGTGTAGTGGTGCTACACTACTTTATAAAGTTC
CAAGGCTCGTTTTCGGTGAAAATGAAACTTTTGTTGGTGCCGAGGATTGG
TTAGAAAAGAGTGGTGTTGAAGTTATCAACGCTCACAATCCTGACTGTAA
GAATTTGATGGATAAGTTCATCAAGGAGAAGCCAGAAGACTGGAACGAAG
ATATTGGTGAATAGGAATTCTCAGA

### Example 5

### Purification and Characterisation of Yeast CDs

To characterize substrate specificities of yeast CDs in more details, proteins were purified using his tagged protein constructs as described in Example 1 (Purification of the recombinant enzymes). According to measurements obtained with Agilent 2100 Bioanalyzer using the manufacturers protocols, D. bruxellensis CD (PZG893) was purified to 90%, while S. cerevisiae CD (PZG738) contained some impurities (FIG. 7).

To characterize the substrate specificities of yeast CDs, purified proteins were measured at 37° C. and fixed cytosine concentration and assays was performed as described in Example 1 (Enzyme assays) (Table 3).

TABLE 1
CD activities with purified his-tagged proteins.

| | Cytosine |
|---|---|
| Protein | (U/mg/min) |
| Sc CD (PZG738) | 888.4 ± 4.8 |
| Db CD (PZG893) | 176.5 ± 5.3 |

The cytosine was tested at a fixed concentration of 500 μM. All assays were performed in triplicates and the results presented are the mean values with standard deviation.

Under these experimental conditions S. cerevisiae CD was much more active compared to the D. bruxellensis CD. Un-optimised temperature or pH of the assay may explain the low activity of D. bruxellensis CD.

### Example 6

### Temperature Stability

Multiple alignment of protein sequences showed that all amino acid residues from S. cerevisiae CD which can lead to higher termostability of the enzyme (Korkegian, A., Black M. E., Baker, D., Stoddard, B. L., (2005) Science 308, 857-860.) were not conserved and it seems that D. bruxellensis wild type enzyme already contains several residues which may give a termostable enzyme (FIG. 4).

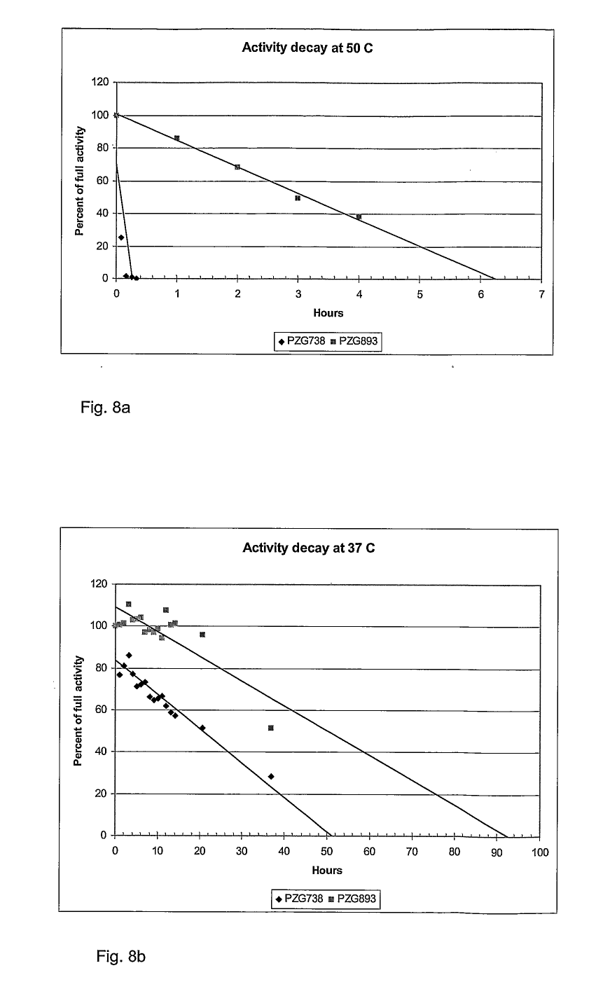

Purified and his-tagged CD from D. bruxellensis and S. cerevisiae CD were assayed after different periods of storage at 50° C. and 37° C. (FIGS. 8a and 8b). Indeed, D. bruxellensis CD was extremely stable at 50° C. when compared to S. cerevisiae CD which lost all activity after only 4 minutes. Half life for D. bruxellensis CD at 50° C. was app. 3 hours (FIG. 8a). When a similar experiment was performed at 37° C. the enzymes had more comparable stability and D. bruxellensis CD was also more stable compared to S. cerevisiae CD at this temperature. It should be observed that the temperature stability of the proteins without his-tags could be different. However, it is assumed that the his-tag does not affect the temperature stability differently for the two enzymes.

Due to the higher temperature stability in particular at 50° C., D. bruxellensis CD is a better enzyme for industrial scale deamination of cytosine derivatives, including 2-thiocytosine, 6-aza-cytosine, 4-azacytosine and 5-FC. Due to the higher stability at body temperature, D. bruxellensis CD is also expected to be superior for therapeutic purposes.

### Example 7

### Cytotoxicity of 5-Fluorocytosine (5-FC)

### Construction of a Retrovirus Vector Expressing CD

ORFs were amplified with Accuzyme DNA polymerase (Bioline) using primers with designed flanking restriction enzyme sites and containing Kozak sequence at 5′ end. D. bruxellensis CD constructs were cloned into the retrovirus vector pLCXSN. The vector is based on pLXSN (Clontech) to which the CMV promoter has been cloned into the polylinker site to form pLCXSN. The constructs obtained was named DbCDCvir (PZG917). pLCXSN alone was used as a control. The plasmids were purified using the Qiagen plasmid kit (QIAGEN) and DNA sequences were verified by DNA sequence determination. HE 293 T packaging cells (ATCC CRL-11268) were cultured at 37° C. in OPTIMEM 1 medium (Life Technologies, Inc.) The constructed retrovirus vectors were transfected into the packaging cells using LipofectAMINE PLUS (Life Technologies, Inc.) according to the protocol provided by the supplier. The medium from the transfected cells was collected 48 hours after transfection, filtered through a 0.45 μm filter, pelleted by ultracentrifugation (50.000×g, 90 minutes at 4° C.) and dissolved in D-MEM.

### Cell Lines and Retroviral Transduction

Cancer cells were purchased from the American Type Culture Collection. Cells were cultured in RPMI, E-MEM or D-MEM with 10% (v/v) Australian originated foetal calf serum and 1 ml/l of gentamicin. Cells were grown at 37° C. in a humidified incubator with a gas phase of 5% CO₂. The cells were transduced with the retrovirus containing medium mixed with 5 μg/ml of Polybrene, incubated for 48 hours and then cultured continuously for 3 weeks in the presence of 300-400 μg/ml Genetecin® (Life Technologies Inc.).

### Cell Proliferation Assay—Cytotoxicity

Cells were plated at densities range of 1.500-3.500 cells/well in 96-well plates coated with poly-L-lysine (Sigma). 5-FC was added after 24 hours of incubation. Each experiment was performed in four replicates. Cell survival was assayed after 96-120 hours of drug exposure by XTT cell proliferation kit (Roche). The data was corrected for background media-only absorbance where after the 50% cell killing drug concentration (IC₅₀ value) was calculated using SigmaPlot® (SPSS Science, Dyrberg Trading, Denmark).

Untransduced U87MG, MCF7, PANC-1 and HT-29 cancer cell lines were tested for sensitivity towards 5-FC. All four cell lines showed the same range of drug sensitivity: IC₅₀ of 2-5 mM.

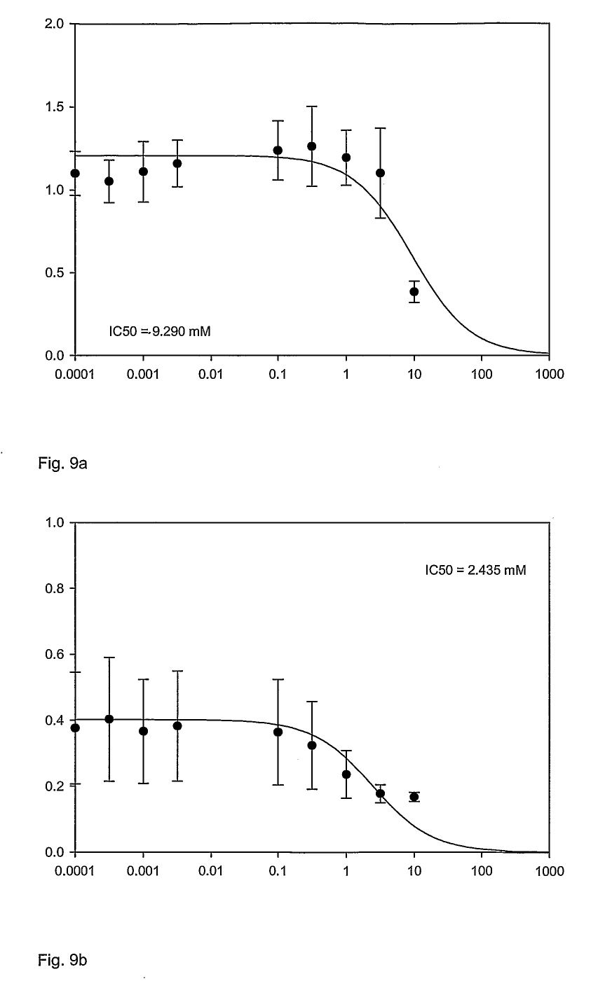

When the MCF7 cell line was transduced with pLCXSN (epmty vector), the IC₅₀ was 9.29 mM (FIG. 9a). When the Db CD gene was trasduced into MCF7 cell line the IC₅₀ was lowered to 2.4 mM, thus leading to a 4 fold sensitivity increase towards 5-FC (FIG. 9b). As can be seen from FIG. 9b the standard deviation of the experiment was relatively high for the cells transduced with D. bruxellensis CD. It is therefore expected that upon replication of the experiment a further lowering of the IC₅₀ may be observed.

The experiment shows that D. bruxellensis cytosine deaminase is capable of increasing the cytotoxicity of 5-FC in human cancer cells and therefore can be used for suicice gene therapy.

1. An isolated cytosine deaminase (EC 3.5.4.1) selected from the group consisting of:
i. a cytosine deaminase derived from Dekkera/Brettanomyces,
ii. a cytosine deaminase comprising an amino acid sequence having at least 70% sequence identity to SEQ ID NO 2, 5 or 8, and
iii. a polypeptide fragment of any of i. through ii. possessing cytosine deaminase activity.

2. The cytosine deaminase of claim 1, comprising an amino acid sequence having at least 70% sequence identity to SEQ ID NO 2.

3. The cytosine deaminase of claim 1, comprising an amino acid sequence having at least 70% sequence identity to SEQ ID NO 5.

4. The cytosine deaminase of claim 1, comprising an amino acid sequence having at least 70% sequence identity to SEQ ID NO 8, more preferably at least 75%, more preferably at least 80%.

5. The cytosine deaminase of claim 1, derived from D. bruxellensis.

6. The cytosine deaminase of claim 1, derived from D. anomala.

7. The cytosine deaminase of claim 1, derived from B. custersianus.

8. The cytosine deaminase of claim 1, comprising the residues marked with a black square in FIG. 6.

9. The cytosine deaminase of claim 1, being able to convert 5-FC into 5-FU.

10. The cytosine deaminase of claim 1, fused to a polypeptide having uracil phosphoribosyltransferase activity.

11. The cytosine deaminase of claim 10, wherein the uracil phosphoribosyltransferase is derived from S. cerevisiae.

12. The cytosine deaminase of claim 1, which when expressed in a cell is capable of reducing the LD₁₀₀ of 5-FC by at least a factor of 2 compared to the LD₁₀₀ for Saccharomyces cerevisiae cytosine deaminase.

13. The cytosine deaminase of claim 12, wherein the LD₁₀₀ is reduced at least by a factor of 4.

14. An isolated nucleic acid molecule selected from the group consisting of:
a. a nucleic acid comprising a cytosine deaminase open reading frame derived from a Dekkera/Brettanomyces species;
b. a nucleic acid comprising a nucleotide sequence being at least 70% identical to SEQ ID NO 1, 4, or 7;
c. a nucleic acid encoding a cytosine deaminase having at least 70% sequence identity to SEQ ID NO 2, 5, or 8;
d. a nucleic acid encoding a cytosine deaminase and being capable of hybridising to a nucleic acid molecule having the complementary sequence of SEQ ID NO 1, 4, or 7;
e. a fragment comprising at least 100 consecutive nucleotide bases of SEQ ID NO 1, 4 or 7; and
f. a subsequence of any of a through d encoding a cytosine deaminase.

15. The nucleic acid of claim 14, comprising a cytosine deaminase open reading frame derived from a Dekkera/Brettanomyces species.

16. The nucleic acid of claim 15, being derived from D. bruxellensis.

17. The nucleic acid of claim 15, being derived from D. anomala.

18. The nucleic acid of claim 15, being derived from B. custersianus.

19. The nucleic acid of claim 14, comprising a nucleotide sequence being at least 70% identical to SEQ ID NO 1.

20. The nucleic acid of claim 14, encoding a cytosine deaminase having at least 70% sequence identity to SEQ ID NO 2.

21. The nucleic acid of claim 14, being capable of hybridising to a nucleic acid molecule having the complementary sequence of SEQ ID NO 1.

22. The nucleic acid of claim 14, comprising a nucleotide sequence being at least 70% identical to SEQ ID NO 4.

23. The nucleic acid of claim 14, encoding a cytosine deaminase having at least 70% sequence identity to SEQ ID NO 5.

24. The nucleic acid of claim 14, being capable of hybridising to a nucleic acid molecule having the complementary sequence of SEQ ID NO 4.

25. The nucleic acid of claim 14, comprising a nucleotide sequence being at least 70% identical to SEQ ID NO 7.

26. The nucleic acid of claim 14, encoding a cytosine deaminase having at least 70% sequence identity to SEQ ID NO 8.

27. The nucleic acid of claim 14, being capable of hybridising to a nucleic acid molecule having the complementary sequence of SEQ ID NO 7.

28. The nucleic acid of claim 21, wherein the hybridisation is under conditions of medium stringency.

29. The nucleic acid of claim 14, being codon optimised for expression in human beings.

30. The nucleic acid of claim 14, having a reduced CpG codon usage.

31. The nucleic acid of claim 14, wherein the nucleic acid is operably fused to a nucleic acid encoding uracil phosphoribosyltransferase.

32. The nucleic acid of claim 31, wherein the uracil phosphoribosyltransferase is derived from a yeast.

33. A vector comprising a nucleic acid according to claim 14.

34. The vector of claim 33, being an expression vector comprising a promoter operably linked to said nucleic acid.

35. The vector of claim 33, wherein the vector is a virus vector.

36. The vector of claim 35, wherein the virus is selected from the group consisting of HIV, SIV, MVA, AAV, AV, and measles virus (MV).

37. An isolated host cell transfected or transduced with the expression vector of claim 34.

38. The host cell of claim 37, being a prokaryotic cell.

39. The host cell of claim 37, wherein the cell is a eukaryotic cell.

40. The host cell of claim 39, being selected from the group consisting of human stem cells and human precursor cells.

41. A process for producing a Dekkera/Brettanomyces cytosine deaminase, comprising culturing a host cell according to claim 37 in vitro and recovering the expressed cytosine deaminase from the culture.

42. A packaging cell line capable of producing an infective vector particle, said vector particle comprising a virally derived genome comprising a 5′ viral LTR, a tRNA binding site, a packaging signal, a promoter operably linked to a polynucleotide sequence encoding a Dekkera/Brettanomyces cytosine deaminase according to claim 1; an origin of second strand DNA synthesis, and a 3′ viral LTR.

43. The packaging cell line according to claim 42, wherein the vector particle is replication defective.

44. The packaging cell line according to claim 43, wherein the genome is lentivirally derived and the LTRs are lentiviral.

45. The packaging cell line according to claim 43, wherein the genome and the LTRs are adeno-associated virus derived.

46. (canceled)

47. (canceled)

48. (canceled)

49. A pharmaceutical composition comprising the polypeptide of claim 1 and a pharmaceutically acceptable diluent, carrier or excipient.

50. The composition of claim 49, further comprising 5-fluorocytosine.

51. A method of treatment of cancer comprising administering to a patient inflicted with cancer a therapeutically effective amount of a Dekkera/Brettanomyces cytosine deaminase according to claim 1 and a therapeutically effective amount of 5-FC.

52. A method of sensitising a mammalian cell to 5-fluorocytosine comprising transfecting said cell with an expression vector according to claim 34, and delivering 5-fluorocytosine to said cell.

53. Use of a polynucleotide sequence encoding a Dekkera/Brettanomyces CD according to claim 1 as a selection marker in molecular biology.

54. A method of deaminating a cytosine derivative, comprising exposing said cytosine derivative to a cytosine deaminase according to claim 1 and recovering the deaminated cytosine derivative.

55. The method of claim 54, wherein the cytosine derivative is selected from the group consisting of 2-thiocytosine, 6-aza-cytosine, 4-aza-cytosine and 5-FC.

56. The method of claim 54, wherein 5-fluorocytosine is subjected to said cytosine deaminase and 5-FU is recovered.

57. The method of claim 54, wherein the process is carried out at a temperature above 35° C.

58. An antibody capable of binding to a CD according to claim 1.

59. A method for controlling the growth of Dekkera/Brettanomyces, which comprises contacting a material comprising or potentially comprising Dekkera/Brettanomyces with a growth-inhibitory amount of 5-fluorocytosine (5-FC).

60. The method of claim 59, wherein the material is a fermented alcoholic beverage and growth is controlled during ageing and/or storage of the fermented alcoholic beverage.

61. The method of claim 60, wherein the alcoholic beverage is wine or beer.

62. The method of claim 60, wherein the 5-FC is added to the beverage before or during ageing or storage.

63. The method of claim 60, wherein the 5-FC is applied to the outside or inside of containers and/or to utensils before or during making and/or ageing and/or storage.

64. The method of claim 63, wherein 5-FC is applied as an aqueous solution to wooden barrels prior to filling with fermented alcoholic beverage.

65. The method of claim 63, wherein 5-FC is applied as an aqueous solution to wood chunks prior to adding the wood chunks to the fermented alcoholic beverage.

66. The method of claim 59, wherein the concentration of 5-FC is below 1 μM.

67. The cytosine deaminase of claim 1, having at least 95% identity to SEQ ID NO 2.

68. The cytosine deaminase of claim 1, having at least 95% identity to SEQ ID NO 5.

69. The cytosine deaminase of claim 1, having at least 95% identity to SEQ ID NO 8.

70. The nucleic acid of claim 14, comprising a nucleotide sequence having at least 95% identity to SEQ ID NO 1.

71. The nucleic acid of claim 14, comprising a nucleotide sequence having at least 95% identical to SEQ ID NO 4.

72. The nucleic acid of claim 14, comprising a nucleotide sequence having at least 95% identity to SEQ ID NO 7.

73. The nucleic acid of claim 14, encoding a cytosine deaminase having at least 95% identity to SEQ ID NO 2.

74. The nucleic acid of claim 14, encoding a cytosine deaminase having at least 95% identity to SEQ ID NO 5.

75. The nucleic acid of claim 14, encoding a cytosine deaminase having at least 95% identity to SEQ ID NO 8.

76. The nucleic acid of claim 28, wherein the hybridisation is under high stringency.

77. A pharmaceutical composition comprising the expression vector of claim 33 and a pharmaceutically acceptable diluent, carrier or excipient.


SEQUENCE LISTING (9 sequences)

SEQ ID NO: 1 - DNA, 453 bp, Dekkera bruxellensis
  CDS (1)..(453)
atgacatttgatgacaaattaggaatgcaggttgccttcgaggaggccaaaaagggatttgaggaaggaggtgtccctattggagcatgtctgcttaccgaggagggaaaggtgattggtcgtggccacaatatgcgtgttcagaagtcatctgccactcttcatggtgaaacatcatgttttgagaatgccggaagattgcccgcttctgtttacaagaaatgcacgctttacaccactttgtctccatgctccatgtgcagtggtgcagccttgttgttcaagattccaaggattgttcttggagaaaacgagacgtttgttggtgcagagaagtggcttgagagtaatggagtggaagttgtgaatgtgcataacaaagagtgcaaaaatctcatggataggtttattaaggagaagccagaggtctggaatgaggatattggcgagtaa

SEQ ID NO: 2 - protein, 150 aa, Dekkera bruxellensis
MTFDDKLGMQVAFEEAKKGFEEGGVPIGACLLTEEGKVIGRGHNMRVQKSSATLHGETSCFENAGRLPASVYKKCTLYTTLSPCSMCSGAALLFKIPRIVLGENETFVGAEKWLESNGVEVVNVHNKECKNLMDRFIKEKPEVWNEDIGE

SEQ ID NO: 3 - DNA, 2707 bp, Dekkera bruxellensis
ggggggggggtctaattgcggtatgctaaattcttgcactgctagttcaattgtgcaacaacgcagatattctctaagtaaatgttttatccgaccaaaaagcccgtcaattctataatatcgacctgtggcctttctctagaatattctcccttcgtttatatatatttgtttcctcttcttcaactcatctcaaactgaaactcgtgcgaacaagtttaaattattattcctattgcaattcagttctgttttttttttactttttcgagttcgtcattcgtgccgcacataatccaaaggggtggctggatcggatcttcctgttgatatggtgtacggatgtgctacgctttcgatgcaagcgggttggactgcatttgtagcagaacaagttatgcaagttatgcaagttataatgccagtttaactaaaagttggcgctatacccatcaaaggcttgatgtcgttgattaacgaataaaataatatccgcaatttgtgtgtgttaatgcatatataaatcgtgcgaaagtcatgcatcaaaattcacaaaacttgagaaggtgagtattatctgaagattaatttaataacaattgggaattaacaatttgacgaagataaataaattgacggaaatagtttaaacgacaacgagcgaaatgcattcaaagtaaatagctgtaataacaattttagagccaacattgcaacatttaacaaccatcatccacatcataaaaagctgtcacaatgcaatcaactcaattagccaactatatgttggcaaaattgcacattttcaagaactttctggaatcgcattagctaaacaatttattctcccatgagttaatcatacatacccttaaactagcacatgaatagatgaagtccctcgcattagtggtcacgtgacataccgcccaacttggaaagtgcagcagataaatcgaaaaaagataaggagcgaggtgaaaattcaacggtggtaaaattttaaatttcacatttctgctatctttcggccccaatactttgaaaagcatctggctgaatatacaacccacacatatttagaactacagaaccgaaattatgacatttgatgacaaattaggaatgcaggttgccttcgaggaggccaaaaagggtaggctgtggagtgtttaagtgcgaaaaatttaacttgtgccccagaataaaacagcattactaacattaagcttgccgattccgctttttttaggatttgaggaaggaggtgtccctattggagcatgtctgcttaccgaggagggaaaggtgattggtcgtggccacaatatgcgtgttcagaagtcatctgccactcttcatggtgaaacatcatgttttgagaatgccggaagattgcccgcttctgtttacaagaaatgcacggtaagtatagtaagagatgcggaaacatgaagcattcttgttgaggtcatgcttttgaactgtgtgtactaaccacagaaccatattcgatacagctttacaccactttgtctccatgctccatgtgcagtggtgcagccttgttgttcaagattccaaggattgttcttggagaaaacgagacgtttgttggtgcagagaagtggcttgagagtaatggagtggaagttgtgaatgtgcataacaaagagtgcaaaaatctcatggataggtttattaaggagaagccagaggtctggaatgaggatattggcgagtaaaaagtctgcgaaagtttatgaaatttggcgaaaggttgtgaagttttgtcagattttgacaatatgtttgacgaaatttacgtaaaagagatgcatgagttggacacacaccaatttttatagaatgtactaggactaatgctagatctgagcacttactggcgaatgagttgatgatttagacagtttgagaccaaaagaagatagttgaggaaaagcgagagtagttgaagattcgatctttatacttggtaaatggtaattgaaatagatcattacattgatgaagattagctaaagagtggatcattacattgatagagataggagtggatcattacattgatagagataggagtggatcattacattgatagagataagagtggatcattacattgatagagataagagtggatcattacatttggcaagaaagtatgagatcaaagacaatcatattgataaagatctaggcttgaatataaacacgtacatccgtaaaaacctgcatcatgtctttcgtgacaacaatataccaactgccaataaagctagaagtggagaaaaaatatacagtgtcagtagaaatacaccattggataatccatttgatttcactttgatttcacttcactcttactcttagcacttgatcagctttacggattactccagtatcaaatgggggtgccatgcgaacaataaaacagtaaaaagtagaacaaataataacggatgaagcccattagcagcggtaagataaggcaagcttgccgaacagtgggaaaggcagtgaaaaaagggaagccaaaaagtcccaagaatactccaacaatgaattttttagttcggcaaattcaccagcgcaaaaaatggaagggcaacagttagcaatgctggcgatattttcaaactagtacaattagacccccccccctcgcttggcatacttctgtgaa

SEQ ID NO: 4 - DNA, 406 bp, Dekkera anomala
  CDS (2)..(406)
tgcaaaaggttataaagaaggtggtgtacccatcggtgcatgcttgttaactgaggagggtaaagttattggtcgtggtcacaatatgagggtgcagaagtcttcaccaattcttcacggagaaacttcttgctacgccaatgcaggaagattacctgctcgtgtttacaagaaatgtactctttacaccaccttgtctccatgctctatgtgtagtggtgctacactactttataaagttccaaggctcgttttcggtgaaaatgaaacttttgttggtgccgaggattggttagaaaagagtggtgttgaagttatcaacgctcacaatcctgactgtaagaatttgatggataagttcatcaaggagaagccagaagactggaatgaagatattggtgaatag

SEQ ID NO: 5 - protein, 134 aa, Dekkera anomala
AKGYKEGGVPIGACLLTEEGKVIGRGHNMRVQKSSPILHGETSCYANAGRLPARVYKKCTLYTTLSPCSMCSGATLLYKVPRLVFGENETFVGAEDWLEKSGVEVINAHNPDCKNLMDKFIKEKPEDWNEDIGE

SEQ ID NO: 6 - DNA, 475 bp, Dekkera anomala
tgcaaaaggttataaagaaggtggtgtacccatcggtaagtataattactatgatttattgctaaacaaatcattactaactgtcattattaggtgcatgcttgttaactgaggagggtaaagttattggtcgtggtcacaatatgagggtgcagaagtcttcaccaattcttcacggagaaacttcttgctacgccaatgcaggaagattacctgctcgtgtttacaagaaatgtactctttacaccaccttgtctccatgctctatgtgtagtggtgctacactactttataaagttccaaggctcgttttcggtgaaaatgaaacttttgttggtgccgaggattggttagaaaagagtggtgttgaagttatcaacgctcacaatcctgactgtaagaatttgatggataagttcatcaaggagaagccagaagactggaatgaagatattggtgaataggaattctcaga

SEQ ID NO: 7 - DNA, 406 bp, Brettanomyces custersianus
  CDS (2)..(406)
tgcaaaaggttataaagaaggtggtatacccatcggtgcatgcttgttaactgaggagggtaaagttattggtcgtggtcacaatatgagggtgcagaagtcttcaccaattcttcacggagaaacttcttgctacgccaatgcaggaagattacctgctcgtgtttacaagaaatgtactctttacaccaccttgtctccatgctctatgtgtagtggtgctacactactttataaagttccaaggctcgttttcggtgaaaatgaaacttttgttggtgccgaggattggttagaaaagagtggtgttgaagttatcaacgctcacaatcctgactgtaagaatttgatggataagttcatcaaggagaagccagaagactggaacgaagatattggtgaatag

SEQ ID NO: 8 - protein, 134 aa, Brettanomyces custersianus
AKGYKEGGIPIGACLLTEEGKVIGRGHNMRVQKSSPILHGETSCYANAGRLPARVYKKCTLYTTLSPCSMCSGATLLYKVPRLVFGENETFVGAEDWLEKSGVEVINAHNPDCKNLMDKFIKEKPEDWNEDIGE

SEQ ID NO: 9 - DNA, 475 bp, Brettanomyces custersianus
tgcaaaaggttataaagaaggtggtatacccatcggtaagtataattactataatttattgctaaacaaatcattactaactgtcattattaggtgcatgcttgttaactgaggagggtaaagttattggtcgtggtcacaatatgagggtgcagaagtcttcaccaattcttcacggagaaacttcttgctacgccaatgcaggaagattacctgctcgtgtttacaagaaatgtactctttacaccaccttgtctccatgctctatgtgtagtggtgctacactactttataaagttccaaggctcgttttcggtgaaaatgaaacttttgttggtgccgaggattggttagaaaagagtggtgttgaagttatcaacgctcacaatcctgactgtaagaatttgatggataagttcatcaaggagaagccagaagactggaacgaagatattggtgaataggaattctcaga